(19) United States
(12) Patent Application Publication
(10) Pub. No.: US 2026/0284078 A1
(43) Pub. Date: Sep. 24, 2026

(54) IMMUNOSUPPRESSIVE REGIMENS AND METHODS THEREOF

(71) Applicant: Biogen MA Inc., Cambridge, MA (US)

(72) Inventors: Dale Morris, Bedford, MA (US); Branka Grubor, Cambridge, MA (US); Fengju Judy Bai, Belmont, MA (US); Linda C. Burkly, Monument Beach, MA (US); Kate Henry, Boston, MA (US)

(21) Appl. No.: 19/474,563

(22) PCT Filed: Apr. 12, 2024
(86) PCT No.: PCT/US24/24340

§ 371 Date: Oct. 10, 2025

Related U.S. Application Data
(60) Provisional application No. 63/459,413, filed on Apr. 14, 2023.

(51) Int. Cl. A61K 31/573; A61K 31/343; A61K 31/436; A61K 31/519; A61K 48/00

(52) U.S. Cl. CPC A61K 31/573; A61K 31/343; A61K 31/436; A61K 31/519; A61K 48/0033

(57) ABSTRACT

Among other things, the present disclosure provides methods, e.g., methods of treating a subject, methods of immuno-suppression, comprising administering a recombinant adeno-associated viral vector (rAAV) and an immunosuppressive regimen. In some embodiments, the present disclosure provides methods of reducing dorsal root ganglion (DRG) toxicity. In some embodiments, the present disclosure provides immunosuppressive regimens comprising, e.g., dexamethasone and one or more immunosuppressive agents, e.g., a calcineurin inhibitor.

### CROSS-REFERENCE TO RELATED APPLICATIONS

This application claims priority to U.S. Provisional Application Ser. No. 63/459,413, filed Apr. 14, 2023, the entirety of which is incorporated herein by reference.

### BACKGROUND

Gene therapy using adeno-associated viral vectors (rAAVs) is an important and developing modality for treating a multitude of diseases, disorders, and conditions. Usage of rAAVs has been marked by a number of challenges including, e.g., toxicity that may affect particular organs, tissues, or cells in a subject administered a rAAV. For example, dorsal root ganglion toxicity (e.g., lesions) related to administration of rAAVs, particularly those targeted to the central nervous system (CNS), is an ongoing challenge. Such toxicity may result from immune responses to one or more components of a rAAV. Accordingly, there remains a need for therapeutic approaches to reduce or ameliorate rAAV-associated toxicity.

### SUMMARY

In some aspects, the present disclosure provides methods of treating a subject, comprising administering a recombinant adeno-associated viral vector (rAAV) and administering an immunosuppressive regimen.

In some embodiments, an immunosuppressive regimen comprises dexamethasone and a calcineurin inhibitor. In some embodiments, a calcincurin inhibitor comprises a macrolide. In some embodiments, a calcineurin inhibitor comprises tacrolimus.

In some embodiments, a rAAV comprises a nucleic acid sequence encoding a polypeptide. In some embodiments, a rAAV comprises a nucleic acid sequence encoding an RNA molecule. In some embodiments, a rAAV comprises a nucleic acid sequence encoding a miRNA. In some embodiments, a rAAV comprises a nucleic acid sequence encoding a polypeptide and a nucleic acid sequence encoding an RNA molecule.

In some embodiments, a rAAV is administered intravenously, intrathecally, intracerebroventricularly, intracisternally, intramuscularly, intraparenchymally, intracranially, intraocularly, intraarticularly, intranasally, and/or subcutaneously. In some embodiments, a rAAV is administered intravenously.

In some embodiments, an immunosuppressive regimen comprises dexamethasone and a calcineurin inhibitor, and one or more additional immunosuppressive agents. In some embodiments, an immunosuppressive regimen comprises dexamethasone and a calcineurin inhibitor, and two or more additional immunosuppressive agents. In some embodiments, an immunosuppressive regimen comprises dexamethasone and a calcineurin inhibitor, and three or more additional immunosuppressive agents. In some embodiments, an immunosuppressive regimen comprises dexamethasone and a calcineurin inhibitor, and an inosine monophosphate dehydrogenase (IMPDH) and/or a Janus kinase (JAK) inhibitor. In some embodiments, an immunosuppressive regimen comprises dexamethasone, a calcineurin inhibitor, and an IMPDH inhibitor. In some embodiments, an immunosuppressive regimen comprises dexamethasone, a calcineurin inhibitor, and a JAK inhibitor. In some embodiments, an immunosuppressive regimen comprises dexamethasone, a calcineurin inhibitor, an IMPDH inhibitor, and a JAK inhibitor. In some embodiments, an IMPDH inhibitor comprises mycophenolate mofetil (MMF). In some embodiments, a JAK inhibitor comprises tofacitinib.

In some embodiments, dexamethasone is administered intraosseously, intrathecally, intravenously, and/or orally. In some embodiments, dexamethasone is administered intraosseously. In some embodiments, dexamethasone is administered intrathecally. In some embodiments, dexamethasone is administered intravenously. In some embodiments, dexamethasone is administered at a dose between about 0.01 mg/kg to about 10 mg/kg, about 0.1 mg/kg to about 5 mg/kg, about 0.25 mg/kg to about 2.5 mg/kg, or about 0.5 mg/kg to about 1.0 mg/kg. In some embodiments, dexamethasone is administered orally. In some embodiments, dexamethasone is administered daily.

In some embodiments, dexamethasone is administered (i) on each of 1, 2, 3, 4, 5, 6, 7, 8, 9, 10, or more days prior to administration of a rAAV, (ii) on a same day as administration of arAAV, and/or (iii) on each day following administration of a rAAV for about 1 week, 2 weeks, 3 weeks, 4 weeks, 1 month, 2 months, 3 months, 4 months, 5 months, 6 months, or longer.

In some embodiments, a calcineurin inhibitor is administered orally and/or intravenously. In some embodiments, a calcineurin inhibitor is administered orally. In some embodiments, a calcineurin inhibitor is administered intravenously. In some embodiments, a calcineurin inhibitor is administered at a dose of between about 0.01 mg/kg to about 10 mg/kg, about 0.1 mg/kg to about 5 mg/kg, about 0.25 mg/kg to about 2.5 mg/kg, or about 0.5 mg/kg to about 1 mg/kg. In some embodiments, a calcineurin inhibitor is administered daily.

In some embodiments, a calcineurin inhibitor is administered (i) on each of 1, 2, 3, 4, 5, 6, 7, 8, 9, 10, or more days prior to administration of a rAAV, (ii) on a same day as administration of a rAAV, and/or (iii) on each day following administration of a rAAV for about 1 week, 2 weeks, 3 weeks, 4 weeks, 1 month, 2 months, 3 months, 4 months, 5 months, 6 months, or longer.

In some embodiments, MMF is administered orally or intravenously. In some embodiments, MMF is administered orally. In some embodiments, MMF is administered intravenously. In some embodiments, MMF is administered at a dose of between about 0.1 mg/kg to about 200 mg/kg, about 1 mg/kg to about 100 mg/kg, about 10 mg/kg to about 75 mg/kg, or about 25 mg/kg to about 50 mg/kg. In some embodiments, MMF is administered daily. In some embodiments, MMF is administered twice daily.

In some embodiments, MMF is administered (i) on each of 1, 2, 3, 4, 5, 6, 7, 8, 9, 10, or more days prior to administration of a rAAV, (ii) on a same day as administration of arAAV, and/or (iii) on each day following administration of a rAAV for about 1 week, 2 weeks, 3 weeks, 4 weeks, 1 month, 2 months, 3 months, 4 months, 5 months, 6 months, or longer.

In some embodiments, tofacitinib is administered orally or intravenously. In some embodiments, tofacitinib is administered orally. In some embodiments, tofacitinib is administered intravenously. In some embodiments, tofacitinib is administered at a dose of between about 0.01 mg/kg to about 10 mg/kg, about 0.1 mg/kg to about 5 mg/kg, about 0.25 mg/kg to about 2.5 mg/kg, or about 0.5 mg/kg to about 1 mg/kg. In some embodiments, tofacitinib is administered daily.

In some embodiments, tofacitinib is administered (i) on each of 1, 2, 3, 4, 5, 6, 7, 8, 9, 10, or more days prior to administration of a rAAV, (ii) on a same day as administration of a rAAV, and/or (iii) on each day following administration of a rAAV for about 1 week, 2 weeks, 3 weeks, 4 weeks, 1 month, 2 months, 3 months, 4 months, 5 months, 6 months, or longer.

In some embodiments, a subject is a mammal. In some embodiments, a subject is a mouse. In some embodiments, a subject is a rat. In some embodiments, a subject is a rabbit. In some embodiments, a subject is a dog. In some embodiments, a subject is a pig. In some embodiments, a subject is a non-human primate. In some embodiments, a subject is a human. In some embodiments, a subject is an adult. In some embodiments, a subject is a child.

In some embodiments, dorsal root ganglion (DRG) toxicity is reduced relative to administration of arAAV without an immunosuppressive regimen. In some embodiments, lesion severity and/or incidence in one or more DRG is reduced relative to administration of an rAAV without an immunosuppressive regimen. In some embodiments, lesion severity in one or more DRG is reduced relative to administration of a rAAV without an immunosuppressive regimen. In some embodiments, lesion incidence in one or more DRG is reduced relative to administration of a rAAV without an immunosuppressive regimen. In some embodiments, axonal degeneration in one or more DRG is reduced relative to administration of a rAAV without an immunosuppressive regimen.

### BRIEF DESCRIPTION OF THE DRAWINGS

FIGS. 1A-1B. Immune cell foci are present in dorsal root ganglia (DRG) of animals administered rAAV. Cynomolgus macaques were administered 3.68×10¹³ genome copies (GC) of rAAV, comprising a nucleic acid sequence encoding a polypeptide, via a single intracisterna magna (ICM) injection. A control group of animals received vehicle (artificial cerebrospinal fluid; aCSF) only. At days 5, 9, 15, and 29 post-rAAV administration, animals were sacrificed for histopathological analysis of DRG. Immunohistochemistry using a monoclonal anti-CD68 antibody (KP1) was conducted to identify CD68+macrophage infiltration. (A) Representative images for each time point are shown. (B) The CD68+ area was quantified using Visiopharm image analysis software. Each column (e.g., 1001, 1002, 1501) represents one animal. The size of each plotted circle represents the CD68+% area. The Y axis of the graph denotes spinal cord level.

FIGS. 2A-2B. Immune cell foci are present in DRG of animals administered rAAV. Cynomolgus macaques were administered 3.68×10¹³ GC of rAAV, comprising a nucleic acid sequence encoding a polypeptide, via a single ICM injection. A control group of animals received vehicle (artificial cerebrospinal fluid; aCSF) only. At days 5, 9, 15, and 29 post-rAAV administration, animals were sacrificed for histopathological analysis of DRG. Immunohistochemistry using a monoclonal anti-CD4 antibody (EPR6855) was conducted to identify CD4+ T cell infiltration. (A) Representative images for each time point are shown. (B) The CD4+ area was quantified using Visiopharm image analysis software. Each column (e.g., 1001, 1002, 1501) represents one animal. The size of each plotted circle represents the CD4+% area. The Y axis of the graph denotes spinal cord level.

FIGS. 3A-3B. Immune cell foci are present in DRG of animals administered rAAV. Cynomolgus macaques were administered 3.68×10¹³ GC of rAAV, comprising a nucleic acid sequence encoding a polypeptide, via a single ICM injection. A control group of animals received vehicle (artificial cerebrospinal fluid; aCSF) only. At days 5, 9, 15, and 29 post-rAAV administration, animals were sacrificed for histopathological analysis of DRG. Immunohistochemistry using a monoclonal anti-CD20 antibody (L26) was conducted to identify CD20+B cell infiltration. (A) Representative images for each time point are shown. (B) The CD20+ area was quantified using Visiopharm image analysis software. Each column (e.g., 1001, 1002, 1501) represents one animal. The size of each plotted circle represents the CD20+% area. The Y axis of the graph denotes spinal cord level.

FIGS. 4A-4B. Immune cell foci are present in DRG of animals administered rAAV. Cynomolgus macaques were administered 3.68×10¹³ GC of rAAV, comprising a nucleic acid sequence encoding a polypeptide, via a single ICM injection. A control group of animals received vehicle (artificial cerebrospinal fluid; aCSF) only. At days 5, 9, 15, and 29 post-rAAV administration, animals were sacrificed for histopathological analysis of DRG. Immunohistochemistry using a monoclonal anti-CD8 antibody (SP57) was conducted to identify CD8+ T cell infiltration. (A) Representative images for each time point are shown. (B) The CD8+ area was quantified using Visiopharm image analysis software. Each column (e.g., 1001, 1002, 1501) represents one animal. The size of each plotted circle represents the CD8+% area. The Y axis of the graph denotes spinal cord level.

FIGS. SA-SB. Immune cell foci are present in DRG of animals administered rAAV. Cynomolgus macaques were administered 3.68×10¹³ GC of rAAV, comprising a nucleic acid sequence encoding a polypeptide, via a single ICM injection. A control group of animals received vehicle (artificial cerebrospinal fluid; aCSF) only. At days 5, 9, 15, and 29 post-rAAV administration, animals were sacrificed for histopathological analysis of DRG. In situ hybridization using a probe to detect NCR1/Nkp46 mRNA was conducted to identify NK cell infiltration. A negative control probe for dapB (bacterial gene) was also used to confirm specificity of the reaction. (A) Representative images for each time point are shown. Arrows point to regions of NCR1/Nkp46+signal. (B) The NCR1/Nkp46+ area was quantified using Visiopharm image analysis software. Each column (e.g., 1001, 1002, 1501) represents one animal. The size of each plotted circle represents the NCR1/Nkp46+% area. The Y axis of the graph denotes spinal cord level.

FIG. 6. Provided technologies can provide reduction of dorsal root ganglion, trigeminal ganglion, and motor neuron toxicity. Cynomolgus macaques were administered 3×10¹³ GC of rAAV, comprising a nucleic acid sequence encoding a polypeptide, via a single ICM injection alone or in combination with an immunosuppression regimen (IS) comprising dexamethasone (intrathecal, once; oral, daily), tacrolimus (oral, daily), and mycophenolate mofetil (MMF) (oral, twice daily). A control group of animals received neither rAAV nor the immunosuppression regimen. At 4 weeks post-rAAV administration, animals were sacrificed for histopathological analysis of lesions in DRG, spinal cord (SC), and trigeminal ganglion (TG). Each column (e.g., 1001, 1002, 1501) represents one animal. For the top five rows, the three bar columns represent, from left to right, tissue collected from the lumbar, thoracic, and cervical levels, respectively. The Y axis of each graph represents severity score (0=no lesion (0% tissue affected); 1=minimal (<10% tissue affected); 2=mild (10-25% tissue affected); 3=moderate (25-50%); 4=marked (50-95%); 5=severe (>95%)).

FIG. 7. Provided technologies can provide reduction of dorsal root ganglion, spinal cord, and sympathetic ganglion toxicity. Cynomolgus macaques were administered 1×10¹³, 3×10¹³, or 1×10¹⁴ GC of rAAV, comprising a nucleic acid sequence encoding a polypeptide, via a single ICM injection alone or in combination with an immunosuppression regimen (IS) comprising dexamethasone (oral, daily), tacrolimus (oral, daily), and MMF (oral, daily). A control group of animals received only a vehicle (artificial cerebrospinal fluid; aCSF) control. Each column (e.g., 1001, 1002, 1501) represents one animal. Each row of graphs represents scored lesion severity for the region (DRG=dorsal root ganglion; SC=spinal cord; SG=sympathetic ganglion) and type of lesion (e.g., neural degeneration, mononuclear cell infiltration) as listed at left. The Y axis of each graph denotes spinal region (cervical, thoracic, lumbar, sacral). Circle size represents lesion severity score (no circle=no lesion (0% tissue affected); 1=minimal (<10% tissue affected); 2=mild (10-25% tissue affected); 3=moderate (25-50%); 4=marked (50-95%); 5=severe (>95%)).

FIG. 8. Provided technologies can provide reduction of dorsal root ganglion, nerve root, spinal cord, and peripheral nerve toxicity. Cynomolgus macaques were administered 2×10¹³, 4×10¹³, or 7.5×10¹³ GC of rAAV, comprising a nucleic acid sequence encoding a miRNA, via a single intrathecal (IT) injection alone or in combination with an immunosuppression regimen comprising dexamethasone, tacrolimus, and tofacitinib (“Triple”), dexamethasone and tacrolimus (“Dex+Tacro”), or dexamethasone and tofacitinib (“Dex+Tofa”). Dexamethasone and tacrolimus were administered orally daily and tofacitinib was administered orally twice daily. A control group of animals received only a vehicle control. Each column (e.g., 1001, 1501, 1502) represents one animal. The first five rows of graphs represents scored lesion severity for the region (DRG=dorsal root ganglion) and type of lesion (e.g., neural degeneration, mononuclear cell infiltration) as listed at left. The Y axis of each graph denotes spinal cord level. Circle size represents lesion severity score (no circle=no lesion (0% tissue affected); 1=minimal (<10% tissue affected); 2=mild (10-25% tissue affected); 3=moderate (25-50%); 4=marked (50-95%); 5=severe (>95%)). The graph in the last row depicts measured levels of phosphorylated neurofilament heavy (pNfH) chain in the cerebrospinal fluid (CSF). The Y axis of the graph represents pg*day/ml.

FIG. 9. Exemplary peripheral drug exposures in blood and plasma. Cynomolgus macaques were administered 5×10¹³ vg/kg of rAAV, comprising a nucleic acid sequence encoding mCherry protein, via a single intravenous (IV) injection in combination with an immunosuppression regimen comprising dexamethasone and tacrolimus (Dex/Tac) (N=3) or an immunosuppression regimen comprising prednisolone (Pred) (N=3). A control group of animals (N=2) received a vehicle control in combination with dexamethasone and tacrolimus. Dexamethasone, tacrolimus, and/or prednisolone were administered orally once per day. Blood and plasma samples were collected from each animal on day 21, at 4 hours post-dosing with the immunosuppression regimen. The X axis of the graph denotes immunosuppression agent (dexamethasone, tacrolimus, or prednisolone); the Y axis of the graph depicts ng/ml of the immunosuppression agent as detected in blood for dexamethasone and prednisolone or in plasma for tacrolimus. Each circle represents data collected from one animal.

FIGS. 10A-10C. Provided technologies can provide reduction of neuronal damage in vivo. Cynomolgus macaques were administered 5×10¹³ vg/kg of rAAV, comprising a nucleic acid sequence encoding mCherry protein, via a single intravenous (IV) injection in combination with an immunosuppression regimen comprising dexamethasone and tacrolimus (Dex/Tac) (N=3) or an immunosuppression regimen comprising prednisolone (Pred) (N=3). A control group of animals (N=2) received a vehicle control in combination with dexamethasone and tacrolimus. Dexamethasone, tacrolimus, and/or prednisolone were administered orally once per day. Blood samples were collected from each animal on day 21, at 4 hours post-dosing with the immunosuppression regimen, and examined for levels of neurofilament subunit NF—H in plasma. (A) NF—H was quantified, using an enzyme-linked immunosorbent assay (ELISA) (Bio-Techne), as a biomarker for neuronal damage. Columns represent average value for each treatment group. Shapes (square, circle, triangle) indicate measured value for individual animals in treatment group. Animal identifiers (e.g., 2001, 2501, 2002) are displayed in legend at right. The X axis of the graph denotes treatment group; the Y axis of the graph depicts picograms (pg) per milliliter (mL) NF—H detected. (B) Representative images of mCherry protein expression (darkly stained areas) are shown. Treatment group is denoted at left. Inset numbers (2001, 2501, 2002, 5001, 5502, 5601) indicate animal from which the pictured dorsal root ganglia (DRG) tissue was collected. Magnification is indicated at top. Rightmost column of images at 40× magnification are zoomed in areas of the images at 10× magnification from 2001 (top) and 5601 (bottom). (C) Animals were sacrificed at end of study and tissues collected and examined for lesions. Treatment groups of animals are indicated at top. Circle size and fill represents lesion severity score, e.g., for animal 5001, infiltrate, mononuclear cell in dorsal root ganglia, sacral was observed as mild; degeneration/necrosis, neuron in dorsal root ganglia, sacral was observed as minimal; and degeneration, nerve fiber in sciatic nerve was not observed.

FIGS. 11A-11D. Provided technologies can provide reduction of increases in liver enzymes upon treatment with AAV. Cynomolgus macaques were administered 5×10¹³ vg/kg of rAAV, comprising a nucleic acid sequence encoding mCherry protein, via a single intravenous (IV) injection in combination with an immunosuppression regimen comprising dexamethasone and tacrolimus (Dex/Tac) (N=3) or an immunosuppression regimen comprising prednisolone (Pred) (N=3). A control group of animals (N=2) received a vehicle control in combination with dexamethasone and tacrolimus. Dexamethasone, tacrolimus, and/or prednisolone were administered orally once per day. Blood samples were collected from animals on days-7, 4, 8, 15, and 22 (wherein 0=day of rAAV administration) and subsequently analyzed for levels of certain liver enzymes and total bilirubin. (A) Level of aspartate transaminase (AST) was quantified. The X axis depicts day of study (wherein 0=day of rAAV administration); the Y axis depicts units (U) per liter (L) of AST detected. Each graphed line corresponds to data collected from one animal. Legend at right identifies treatment group. From top to bottom at study day=15: top three lines correspond to animals treated with rAAV+prednisolone; next three lines correspond to animals treated with rAAV+dexamethasone/tacrolimus; and bottom two lines correspond to animals treated with vehicle+dexamethasone/tacrolimus. (B) Level of alanine transaminase (ALT) was quantified. The X axis depicts day of study (wherein 0=day of rAAV administration); the Y axis depicts units (U) per liter (L) of ALT detected. Each graphed line corresponds to data collected from one animal. Legend at right identifies treatment group. From top to bottom at study day=22: top three lines correspond to animals treated with TAAV+prednisolone; next three lines correspond to animals treated with rAAV+dexamethasone/tacrolimus; and bottom two lines correspond to animals treated with vehicle+dexamethasone/tacrolimus. (C) Level of gamma-glutamyl transferase (GGT) was quantified. The X axis depicts day of study (wherein 0=day of rAAV administration); the Y axis depicts units (U) per liter (L) of GGT detected. Each graphed line corresponds to data collected from one animal. Legend at right identifies treatment group. From top to bottom at study day=15: top three lines correspond to animals treated with rAAV+prednisolone; next one line corresponds to an animal treated+with rAAV+dexamethasone/tacrolimus; next one line corresponds to an animal treated with vehicle+dexamethasone/tacrolimus; next one line corresponds to an animal treated with rAAV+dexamethasone/tacrolimus; next one line corresponds to an animal treated with vehicle+dexamethasone/tacrolimus; and bottom line corresponds to an animal treated with rAAV+dexamethasone/tacrolimus. (D) Level of total bilirubin (TBIL) was quantified. The X axis depicts day of study (wherein 0=day of rAAV administration); the Y axis depicts milligrams (mg) per deciliter (dL) of total bilirubin. Each graphed line corresponds to data collected from one animal. Legend at right identifies treatment group. From top to bottom at study day=15: top three lines correspond to animals treated with rAAV+prednisolone; next one line corresponds to an animal treated with vehicle+dexamethasone/tacrolimus; next three lines correspond to animals treated with rAAV+dexamethasone/tacrolimus; and bottom line corresponds to an animal treated with vehicle+dexamethasone/tacrolimus.

FIG. 12. Provided technologies can provide reduction of liver toxicity. Cynomolgus macaques were administered 5×10¹³ vg/kg of rAAV, comprising a nucleic acid sequence encoding mCherry protein, via a single intravenous (IV) injection in combination with an immunosuppression regimen comprising dexamethasone and tacrolimus (Dex/Tac) (N=3) or an immunosuppression regimen comprising prednisolone (Pred) (N=3). A control group of animals (N=2) received a vehicle control in combination with dexamethasone and tacrolimus. Dexamethasone, tacrolimus, and/or prednisolone were administered orally once per day. Animals were sacrificed on day 22 and livers collected and examined for lesions. Treatment groups of animals are indicated at top. None of the listed liver lesions were observed in animals treated with vehicle in combination with dexamethasone and tacrolimus. Circle size and fill represents lesion severity score, e.g., for animal 5001, apoptosis/single cell necrosis was observed as mild whereas hyperplasia, bile duct was observed as minimal.

### DEFINITIONS

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Adeno-associated virus (AAV): As used herein, the terms “Adeno-associated virus” and “AAV” refer to viral particles, in whole or in part, of family Parvoviridae and genus Dependoparvovirus. AAV is a small, replication-defective, non-enveloped virus. AAV may include, but is not limited to, AAV serotype 1, AAV serotype 2, AAV serotype 3 (including serotypes 3A and 3B), AAV serotype 4, AAV serotype 5, AAV serotype 6, AAV serotype 7, AAV serotype 8, AAV serotype 9, AAV serotype 10, AAV serotype 11, AAV serotype 12, AAV serotype 13, AAV serotype rh10, AAV serotype rh74, AAV from the HSC 1-17 series, AAV from the CBr, CLv or CLg series, snake AAV, avian AAV, bovine AAV, canine AAV, equine AAV, ovine AAV, goat AAV, shrimp AAV, and any variant of any of the foregoing. AAV may also include engineered or chimeric versions of a wild-type AAV that include one or more insertions, deletions and/or substitutions within the Cap polypeptide(s) that affect one or more properties of the wild-type AAV serotype, including without limitation tropism and evasion of neutralizing antibodies (e.g., AAV-DJ, AAV-PHP.B, AAV-PHP.N, AAV.CAP-B1 to AAV.CAP-B25 and variants thereof). Wild-type AAV is replication deficient and requires co-infection of cells by a helper virus (e.g., adenovirus, herpes, or vaccinia virus) or supplementation of helper viral genes in order to replicate.

Administration: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, subretinal, topical, tracheal (including by intratracheal instillation), transdermal, vaginal, vitreal, or any combination thereof. In some embodiments, administration may be subretinal. In some embodiments, a preferred method of administration will reduce or prevent an immune response from a subject receiving treatment.

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present disclosure include small molecules, antibodies, antibody fragments, aptamers, siRNAs, shRNAs, miRNAs, DNA/RNA hybrids, antisense oligonucleotides, ribozymes, peptides, peptide mimetics, small molecules, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Comprising: A composition or method described herein as “comprising” or “including” one or more named elements or steps is open-ended, meaning that the named elements or steps are essential, but other elements or steps may be added within the scope of the composition or method. To avoid prolixity, it is also understood that any method described as “comprising” (or which “comprises”) one or more named elements or steps also describes the corresponding, more limited method “consisting essentially of” (or which “consists essentially of”) the same named elements or steps, meaning that the method includes the named essential elements or steps and may also include additional elements or steps that do not materially affect the basic and novel characteristic(s) of the method. It is also understood that any method described herein as “comprising” or “consisting essentially of” one or more named elements or steps also describes the corresponding, more limited, and closed-ended method “consisting of” (or “consists of”) the named elements or steps to the exclusion of any other unnamed element or step. In any method disclosed herein, known or disclosed equivalents of any named essential element or step may be substituted for that element or step.

Immunosuppressive agent: As used herein, the term “immunosuppressive agent,” in its broadest sense, refers to an agent, e.g., a therapeutic agent that suppresses or reduces the activation, activity, or efficacy of the immune system of a subject. Exemplary immunosuppressive agents include, but are not limited to, abatacept, abrocitinib, adalimumab, alemtuzumab, anakinra, atacicept, azathioprine, baricitinib, basiliximab, belatacept, belimumab, bortezomib, certolizumab, crovalimab, cyclophosphamide, cyclosporine, daclizumab, dexamethasone, eculizumab, efalizumab, epratuzumab, etanercept, everolimus, fingolimod, fluorouracil, golimumab, hydroxychloroquine, imlifidase, infliximab, leflunomide, mercaptopurine, methotrexate, methylprednisolone, mycophenolate mofetil, mycophenolate sodium, ocrelizumab, ofatumumab, pimecrolimus, prednisone, prednisolone, ridaforolimus, rilonacept, rituximab, ruxolitinib, secukinumab, sirolimus, tacrolimus, temsirolimus, tocilizumab, tofacitinib, upadacitinib, and veltuzumab. Additional immunosuppressive agents are known in the art.

Immunosuppressive regimen: As used herein, the term “immunosuppressive regimen,” in its broadest sense, refers to a treatment regimen comprising one or more immunosuppressive agents.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester scaffold. For example, in some embodiments, a nucleic acid is, comprises, or consists of one or more “peptide nucleic acids”, which are known in the art and have peptide bonds instead of phosphodiester bonds in the scaffold, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosinc, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid can comprise or consist of one or more inhibitory nucleic acids (e.g., small RNA molecules). In some embodiments, an inhibitory nucleic acid comprises or consists of an RNA molecule (e.g., a small RNA molecule) that inhibits gene expression (e.g., via mRNA degradation) or inhibits translation (e.g., decreases the level of gene expression or translation of a transcript as compared to a relevant control). In some embodiments, an inhibitory nucleic acid comprises or consists of one or more siRNA, miRNA, shRNA, gRNA, or any combination thereof. In some embodiments, an inhibitory nucleic acid can be single stranded or double stranded.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, a pharmaceutical composition is formulated for subretinal administration, e.g., by subretinal injection.

Polypeptide: As used herein, the term “polypeptide” refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide has an amino acid sequence encoded by a sequence that does not occur in nature (e.g., a sequence that is engineered in that it is designed and/or produced through action of the hand of man to encode said polypeptide). In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Subject: As used herein, the terms “subject” or “patient,” as used interchangeably herein, refers to any organism to which a provided composition is or may be administered, e.g., for experimental, diagnostic, prophylactic, cosmetic, and/or therapeutic purposes. In some embodiments, a subject is or comprises a cell or a tissue. Typical patients include animals (e.g., mammals, such as mice, rats, rabbits, non-human primates, and/or humans). In some embodiments, a patient is a human. In some embodiments, a patient is suffering from or susceptible to one or more diseases, disorders, or conditions. In some embodiments, a patient displays one or more symptoms of a disease, disorder, or condition. In some embodiments, a patient has been diagnosed with one or more diseases, disorders, or conditions.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, or urine). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Treat: As used herein, the terms “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into a viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In some embodiments, the term “vector” refers to an agent capable of transporting a nucleic acid, wherein the agent comprises the nucleic acid. In some embodiments, a vector comprises or is an agent capable of transporting a nucleic acid.

### DETAILED DESCRIPTION OF CERTAIN EMBODIMENTS

rAAVs have become an increasingly important method of delivery for gene therapies. However, the implementation of rAAVs has been marked by numerous challenges, including rAAV-associated toxicity in particular organs, tissues, or cells. Usage of rAAVs for targeting of the central nervous system (CNS) has been particularly marked by reported toxicity in the dorsal root ganglia that may in part be due to immune system responses to one or more components of such rAAVs. Accordingly, technologies, e.g., rAAVs, immunosuppressive agents, immunosuppressive regimens, and methods thereof, are needed to address rAAV-associated toxicity including that toxicity that has been reported in the DRG. Among other things, the present disclosure provides various rAAVs, immunosuppressive agents, immunosuppressive regimens, and methods thereof. In some embodiments, immunosuppressive regimens comprise one or more immunosuppressive agents, e.g., dexamethasone and/or a calcineurin inhibitor, such as a macrolide (e.g., tacrolimus). In some embodiments, the present disclosure provides methods comprising administering a rAAV and an immunosuppressive regimen to a subject. In some embodiments, rAAVs, immunosuppressive agents, and/or immunosuppressive regimens are administered as described herein. In some embodiments, provided technologies (e.g., immunosuppressive agents, immunosuppressive regimens, methods thereof) may reduce dorsal root ganglion (DRG) toxicity, e.g., lesion severity and/or incidence, axonal degeneration.

Recombinant Adeno-Associated Virus (rAAV)

AAV is reportedly a small, non-enveloped virus that packages a single-stranded, linear DNA genome of approximately 4.7-5 kb long. A member of the family Parvoviridae, AAV was discovered in 1965 as a contaminant of adenovirus isolates. AAV has not been associated with any human or animal disease, even though most humans (>70%) are seropositive for one or more serotypes (Calcedo et al. (2011); Calcedo et al. (2009), each of which is hereby incorporated by reference in their entirety). Both positive and negative DNA strands are packaged equally well, and infection can be initiated with particles containing either strand. The virus has a T=1 icosahedral capsid, 25 nm in diameter, that is reportedly extraordinarily stable. AAV has been demonstrated to resist brief exposure to heat, acidic pH, and proteases. An AAV genome comprises three open reading frames (ORFs), rep (replication), cap (capsid), and aap (assembly-activating protein), which together code for eight proteins (Rep78, Rep68, Rep52, Rep40, VP1, VP2, VP3, and AAP) expressed from three promoters (p5, p19, and p40). The mature capsid comprises the amino acid sequence of only one ORF (cap) and the packaged DNA.

In some embodiments, an AAV is a recombinant AAV (rAAV). rAAV vectors have become widely used for inserting genes into mammalian cells (e.g., human cells). rAAV systems are generally well known in the art (see, e.g., Kelleher and Vos, Biotechniques, 17 (6): 1110-17 (1994); Cotten et al., P.N.A.S. U.S.A., 89 (13): 6094-98 (1992); Curiel, Nat Immun, 13 (2-3): 141-64 (1994); Muzyczka, Curr Top Microbiol Immunol, 158:97-129 (1992); and Asokan et al., Mol. Ther., 20 (4): 699-708 (2012), each of which is hereby incorporated by reference in its entirety). Methods for generating and using rAAV vectors are described, for example, in U.S. Pat. Nos. 5,139,941 and 4,797,368, each of which is hereby incorporated by reference in its entirety.

In some embodiments, a rAAV comprises or is a naturally occurring AAV. In some embodiments, a rAAV is a modified AAV or a variant of a naturally occurring AAV. In some embodiments, a rAAV is generated by directed evolution, e.g., by DNA shuffling, peptide insertion, or random mutagenesis, in order to introduce modifications into the AAV sequence to improve one or more properties for gene therapy. In some embodiments, such modifications avoid or lessen an immune response or recognition by neutralizing antibodies and/or allow for more efficient and/or targeted transduction (see, e.g., Asuri et al., Molecular Therapy, 20.2:329-338 (2012), which is hereby incorporated by reference in its entirety). In some embodiments, such modifications allow for identification of AAVs with modified tropism. Methods of using directed evolution to engineer a rAAV can be found, e.g., in U.S. Pat. No. 8,632,764, which is hereby incorporated by reference in its entirety. In some embodiments, a modified AAV is modified to include a specific tropism.

In some embodiments, a rAAV is derived from an AAV genome sequence or a variant thereof as described in U.S. Pat. Nos. 7,906,111; 6,759,237; 7,105,345; 7,186,552; 9,163,260; 9,567,607; 4,797,368; 5,139,941; 5,252,479; 6,261,834; 7,718,424; 8,507,267; 8,846,389; 6,984,517; 7,479,554; 6,156,303; 8,906,675; 7,198,951; 10,041,090; 9,790,472; 10,308,958; 10,526,617; 7,282,199; 7,790,449; 8,962,332; 9,587,250; 10,590,435; 10,265,417; 10,485,883; 7,588,772; 8,067,01; 8,574,583; 8,906,387; 8,734,809; 9,284,357; 10,035,825; 8,628,966; 8,927,514; 9,623,120; 9,777,291; 9,783,825; 9,803,218; 9,834,789; 9,839,696; 9,585,971; or 10,519,198; U.S. Publication Nos. 2017/0166926; 2019/0015527; 2019/0054188; or 2020/0080109; or International Publication Nos. WO2018/160582, WO2020/028751, or WO2020/068990, each of which is hereby incorporated by reference in its entirety.

In some embodiments, a rAAV vector is a dual or triple rAAV vector, e.g., for delivery of large payloads (e.g., payloads of greater than approximately 5 kb) and/or to address safety concerns associated with administration of single rAAV vectors. In some embodiments, a dual rAAV vector includes two separate rAAV vectors, each including a fragment of a full sequence of a large payload of interest, and when recombined, the fragments form the full sequence of the large payload of interest or a functional portion thereof. In some embodiments, a triple rAAV vector includes three separate rAAV vectors, each including a fragment of a sequence of a large payload of interest, and when recombined, the fragments form the full sequence of the large payload of interest or a functional portion thereof.

Multiple rAAV vectors (e.g., dual or triple rAAV vectors) can be delivered to and co-transduced into the same cell, where fragments of a payload of interest recombine and generate a single mRNA transcript of the entire payload of interest. In some embodiments, fragmented payloads include a non-overlapping sequences. In some embodiments, fragmented payloads include a specified overlapping sequences. In some embodiments, multiple rAAV vectors for dual or triple transfection may be the same type of rAAV vector (e.g., same serotype and/or same construct). In some embodiments, multiple rAAV vectors of dual or triple transfection may be different types of rAAV vector (e.g., different serotype or different construct).

In some embodiments, a rAAV vector comprises a single-stranded (ss) or self-complementary (sc) rAAV nucleic acid vector. In some embodiments, a rAAV vector comprises an expression construct and one or more regions comprising ITR sequences (e.g., wild-type ITR sequences or engineered ITR sequences) flanking an expression construct. In some embodiments, a rAAV vector is encapsidated by a viral capsid. In some embodiments, a viral capsid comprises 60 capsid protein subunits. In some embodiments, a viral capsid comprises VP1, VP2, and VP3. In some embodiments, VP1, VP2, and VP3 subunits are present in a capsid at a ratio of about 1:1:10, respectively.

### AAV Serotypes

rAAVs for use in methods described herein may be of any AAV serotype. AAV serotypes generally have different tropisms to target different tissues. In some embodiments, a rAAV serotype is selected based on a tropism. Several AAV serotypes have been characterized including, but not limited to, AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, AAV-PHP.eB, AAV2-retro, AAV9-retro, or AAV.CAP-B1 to AAV.CAP-B25, as well as variants or hybrids thereof. In some embodiments, a rAAV serotype is a variant generated using directed evolution. A rAAV of a particular serotype may comprise ITRs derived from a different serotype. For example, a rAAV may comprise or is an AAV2/5, AAV2/6, AAV2/8 or AAV2/9 vector (e.g., AAV6, AAV8 or AAV9 serotype having AAV2 ITR)

In some embodiments, an AAV serotype has or comprises a mutation in a wild-type AAV sequence. For example, an AAV serotype may have or comprise a mutation in an AAV9 sequence (e.g., as described in Pulicherla et al., Molecular Therapy, 19 (6): 1070-1078 (2011), which is hereby incorporated by reference in its entirety). ArAAV serotype (with corresponding nucleotide and amino acid substitutions) may include, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (CI 531 A, T1609A; L5111, L537M), AAV9.55 (T1605A; F535L), AAV9.58 (C1475T, C1579A; T4921, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L), and AAV9.95 (T1605A; F535L). In certain embodiments, an AAV9 variant comprises or is AAVhu68 or a variant thereof (e.g., as described in International Publication No. WO2018/160585, which is hereby incorporated by reference in its entirety). Other rAAVs are described in, e.g., Sharma et al., Brain Res Bull., 81 (2-3): 273 (2010), which is hereby incorporated by reference in its entirety.

An AAV serotype may be from any number of species. For example, a rAAV may be or comprise an avian AAV (AAAV), e.g., as described in U.S. Pat. No. 9,238,800, which is hereby incorporated by reference in its entirety. A rAAV serotype may be or comprise a bovine AAV (BAAV), e.g., as described in U.S. Pat. Nos. 9,193,769 or 7,427,396, each of which is hereby incorporated by reference in its entirety. A rAAV may be or comprise a caprine AAV, e.g., as described in U.S. Patent No. 7,427,396, which is hereby incorporated by reference in its entirety. A rAAV serotype may also be a variant or hybrid of any of the foregoing.

A rAAV may comprise or be based on a serotype selected from any following serotypes or variants thereof including, but not limited to, AAV9.68, AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb. 1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.84, AAV9.9, AAVA3.3, AAVA3.4, AAVA3.5, AAV A3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.l, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu. 10, AAVhu.11, AAVhu. 12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC 8, AAVpi.1, AAVpi.2, AAVpi.3, AAVrh.10, AAVrh.12, AAVrh. 13, AAVrh. 13R, AAVrh.14, AAVrh.17, AAVrh. 18, AAVrh. 19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, B P61 AAV, B P62 AAV, B P63 AAV, bovine AAV, caprine AAV, Japanese AAV10, true type AAV (ttAAV), UPENN AAV 10, AAV-LK 16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and AAV SM 10-8.

### Capsids

The present disclosure encompasses the recognition that more than 110 distinct primate AAV capsid sequences have been reportedly isolated. Each of those AAV capsids that have unique serological profiles has been named as a particular AAV serotype. The present disclosure further appreciates that at least 12 primate serotypes (e.g., AAV1, AAV2, AAV3, AAV4, AAV5, AAV6, AAV7, AAV8, AAV9, AAV10, AAV11, AAV12) have been described. In some embodiments of the present disclosure, a capsid from any serotype can be used. In some embodiments, a modified or engineered capsid including, but not limited to those described herein, can be used in accordance with the present disclosure.

The present disclosure additionally encompasses the recognition that it may be advantageous to modify wild type AAV capsids, or engineer AAV capsids, to achieve modified tissue tropism and/or immune system evasion. One method of achieving these advantages is to produce a vector in the presence of cap genes for multiple serotypes. Depending on the ratio of capsid proteins from each serotype, the resulting “mosaic” virions can exhibit a combined tropism for cell type or, in some cases, can acquire tropism not exhibited by either serotype individually. Some studies have involved attaching exogenous molecules to the capsid. One example utilizes a bi-specific antibody obtained by fusing Fc regions of two different antibodies: an anti-capsid antibody and an anti-cell marker antibody, thereby conferring rAAV2 tropism to transduction-resistant megakaryocyte cell lines. Another example adopted the approach of biotinylating the capsid and subsequently binding it to a streptavidin conjugate carrying epidermal growth factor or fibroblast growth factor. This approach was shown to produce at least a ten-fold increase in the transduction of cells that highly express the epidermal growth factor or fibroblast growth factor receptor, respectively.

The present disclosure also appreciates that as an alternative to attaching molecules to a capsid surface, it may be advantageous to engineer a modification directly into cap gene. As one non-limiting example, green fluorescent protein (GFP) (238 amino acids) can be inserted into AAV2 VP1 and VP2. Although the transduction efficiencies of VP1-GFP and VP2-GFP vectors were 3 and 5 orders of magnitude lower, respectively, than the efficiency of wild-type capsid, the transduction in Hela cells did occur, suggesting a tolerance for inserted sequences in capsid proteins. As another non-limiting example, for modifying cap genes for tissue targeting, a number of researchers have inserted peptide sequences on the basis of known ligand-receptor interactions, or have selected for peptides in phage-display libraries. Another strategy has been to insert random sequences of amino acids, followed by in vitro selection of the best performing capsids. Instead of introducing target-specific peptides, some experiments modified the capsids generically, pending subsequent modification toward targets of choice. For example, a binding site for the Fc portion of antibodies was inserted into the capsid, followed by binding of different antibodies specific for receptors of various cell lines. Another such modification is to insert a biotin-binding site into the capsid, thereby facilitating metabolic biotinylation and allowing flexible targeting with any avidin-conjugated ligands. Some experiments have taken advantage of peptide insertion as well as mosaic capsids with a virion containing both wild-type capsid proteins and engineered capsid proteins, or a virion containing a combination of multiple different modified capsid proteins. Other techniques are under investigation with a view to evading the immune system, and these include coating capsids with polymer.

### Inverted Terminal Repeats (ITRs)

The present disclosure recognizes that AAV coding regions are flanked by inverted terminal repeats (ITRs) that are typically 145 bases long in wild-type AAVs and have a complex T-shaped structure. These repeats are the origins for DNA replication and serve as the primary packaging signal (McLaughlin et al., 1988; Hauswirth et al., 1977, each of which is hereby incorporated by reference in its entirety). The present disclosure further recognizes that ITRs are the only cis-active sequences required for making rAAVs and the only AAV-encoded sequences present in AAV vectors (McLaughlin et al. (1988); Samulski et al. (1989), each of which is hereby incorporated by reference in its entirety). Although AAV ITRs have enhancer activity in the presence of Rep protein, they have minimal promoter or enhancer activity in the absence of Rep protein. Thus, transgenes cloned into an AAV vector must be engineered with an appropriate enhancer, promoter, polyadenylation signal, and/or splice sites to ensure correct gene expression.

ITR sequences of a rAAV can be derived from any rAAV serotype (e.g., AAV1, AAV2, AAV3A, AAV3B, AAV4, AAV5, AAV6, AAV7, AAV8, AAV9, AAV10, AAV11, AAVrh10, AAVrh74, AAV-HSC 1-17, AAV-CBr, AAV-CLv, AAV-CLg, AAV-DJ, AAV-PHP.B, AAV-PHP.N, or AAV.CAP-B1 to AAV.CAP-B25, or variants or hybrids thereof). In some embodiments, ITRs of the present disclosure may comprise engineered or modified ITRs using methods known in the art. In some embodiments, ITRs of the present disclosure may comprise one or more sequence modifications (e.g., deletions or substitutions) as compared to a wild-type ITR sequence.

In some embodiments, ITR sequences are derived from one or more other serotypes, e.g., as described in U.S. Pat. Nos. 7,906,111; 6,759,237; 7,105,345; 7,186,552; 9,163,260; 9,567,607; 4,797,368; 5,139,941; 5,252,479; 6,261,834; 7,718,424; 8,507,267; 8,846,389; 6,984,517; 7,479,554; 6,156,303; 8,906,675; 7,198,951; 10,041,090; 9,790,472; 10,308,958; 10,526,617; 7,282,199; 7,790,449; 8,962,332; 9,587,250; 10,590,435; 10,265,417; 10,485,883; 7,588,772; 8,067,01; 8,574,583; 8,906,387; 8,734,809; 9,284,357; 10,035,825; 8,628,966; 8,927,514; 9,623,120; 9,777,291; 9,783,825; 9,803,218; 9,834,789; 9,839,696; 9,585,971; or 10,519,198; U.S. Publication Nos. 2017/0166926; 2019/0015527; 2019/0054188; or 2020/0080109; or International Publication Nos. WO2018/160582, WO2020/028751, or WO2020/068990, each of which is hereby incorporated by reference in its entirety.

ITR sequences and plasmids containing ITR sequences are known in the art and are commercially available (See, e.g., products and services available from Vector Biolabs, Philadelphia, PA; Cellbiolabs, San Diego, CA; Agilent Technologies, Santa Clara, CA; and Addgene, Cambridge, MA; and described in Kessler et al., PNAS, 93 (24): 14082-7 (1996); Machida, Methods in Molecular Medicine, Viral Vectors for Gene Therapy Methods and Protocols. 10.1385/1-59259-304-6:201, Humana Press Inc. 2003. Chapter 10. Targeted Integration by Adeno-Associated Virus; and U.S. Pat. Nos. 5,139,941 and 5,962,313; each of which is hereby incorporated by reference in its entirety).

### Production

Methods of producing and isolating rAAV with a desired isolated nucleic acid sequence or vector, and capsid are well known in the art. rAAVs can be produced and isolated according to any appropriate method, e.g., methods described in Clément and Grieger, 2016, Grieger et al., 2016, and Martin et al., 2013, the contents of each which are incorporated herein by reference in their entirety. Without wishing to be bound by any particular theory or process, production methods typically involve culturing a host cell which contains a nucleic acid sequence (e.g., a cap gene) encoding an AAV capsid protein or fragment thereof; a functional rep gene; a nucleic acid sequence or vector comprising AAV ITRs (e.g., an AAV 5′ ITR and an AAV 3′ ITR) and a nucleic acid sequence encoding a product of interest (e.g., a polypeptide, e.g., a wild-type polypeptide); and sufficient helper functions to permit packaging of recombinant AAV vector into the AAV capsid proteins.

The components to be cultured in a host cell to package an isolated nucleic acid sequence or vector in an AAV capsid may be provided to a host cell in trans. Alternatively, any one or more required components (e.g., isolated nucleic acid sequence or vector, rep sequences, cap sequences, and/or helper functions) may be provided by a stable host cell which has been engineered to contain one or more of the required components using methods known to those of skill in the art. Most suitably, such a stable host cell will contain a required component or components under control of an inducible promoter. However, such required component or components may be under the control of a constitutive promoter. Examples of suitable promoters are provided herein. In still another alternative, a selected stable host cell may contain a selected component or components under control of a constitutive promoter and other selected component or components under control of one or more inducible promoters. For example, a stable host cell may be generated which is derived from 293 cells (which contain E1 helper functions under the control of a constitutive promoter), but which contain rep and/or cap proteins under control of inducible promoters. Still other stable host cells are known in the art or may be generated by one of skill in the art.

The isolated nucleic acid sequence or vector, rep sequences, cap sequences, and helper functions required for producing rAAVs of the disclosure may be delivered to a packaging host cell using any appropriate genetic element (e.g., a vector). The selected genetic element may be delivered by any suitable method (e.g., transfection), including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and selection of a suitable method is not a limitation on the present disclosure. See, e.g., K. Fisher et al., 1993 and U.S. Pat. No. 5,478,745.

In some embodiments, rAAVs may be produced using a triple transfection method (e.g., as described in detail in U.S. Pat. No. 6,001,650, the contents of which relating to the triple transfection method are incorporated herein by reference). Typically, such rAAVs are produced by transfecting a host cell with a suitable vector (comprising a nucleic acid sequence encoding a product of interest, e.g., a polypeptide) to be packaged into rAAV particles, an AAV rep/cap vector, and a helper function vector. An AAV rcp/cap vector encodes rep and cap sequences, which function in trans for productive AAV replication and encapsidation. In some embodiments, an AAV rep/cap vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. A helper function vector encodes nucleotide sequences for non-AAV derived viral and/or cellular functions upon which AAV is dependent for replication (e.g., “helper functions”). Helper functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based helper functions can be derived from any of the known helper viruses such as adenovirus, herpesvirus (other than herpes simplex virus type-1), and vaccinia virus.

Additional methods for generating and isolating rAAVs are described in, e.g., U.S. Pat. Nos. 7,790,449; 7,282,199; International Publication Nos. WO2003/042397, WO2005/033321, and WO2006/110689; and U.S. Pat. No. 7,588,772, each of which are hereby incorporated by reference in their entirety.

### Immunosuppressive Agents and Regimens

The present disclosure, among other things, provides immunosuppressive agents and regimens for administration with rAAV. Various immunosuppressive agents are known in the art. Immunosuppressive agents may target a variety of immune system components including, e.g., T cells, B cells, cytokines, or chemokines. Additionally, immunosuppressive agents may target a variety of molecular signaling pathways including, e.g., calcineurin signaling pathway, JAK/STAT signaling pathway, mTOR signaling pathway, and/or TNF signaling pathway. Some immunosuppressive agents target metabolic processes including, e.g., purine biosynthesis and/or pyrimidine biosynthesis. Immunosuppressive agents may comprise a variety of structures including, e.g., an antibody, a steroid, a macrolide, a purine analogue, or a pyrimidine analogue. Any suitable immunosuppressive agent may be used in immunosuppressive regimens as described herein. Any suitable immunosuppressive agent may be used in methods described herein.

In some embodiments, one or more immunosuppressive agents comprise an alkylating agent. In some embodiments, one or more immunosuppressive agents comprise an antimetabolite. In some embodiments, one or more immunosuppressive agents comprise a B cell inhibitor. In some embodiments, one or more immunosuppressive agents comprise a calcincurin inhibitor. In some embodiments, one or more immunosuppressive agents comprise a complement inhibitor. In some embodiments, one or more immunosuppressive agents comprise a cytostatic. In some embodiments, one or more immunosuppressive agents comprise an interleukin-1 receptor antagonist. In some embodiments, one or more immunosuppressive agents comprise an inosine-5′-monophosphate dehydrogenase (IMPDH) inhibitor. In some embodiments, one or more immunosuppressive agents comprise an interleukin-1 receptor-associated kinase 4 (IRAK4) inhibitor. In some embodiments, one or more immunosuppressive agents comprise a Janus kinase (JAK) inhibitor. In some embodiments, one or more immunosuppressive agents comprise a mammalian target of rapamycin (mTOR) inhibitor. In some embodiments, one or more immunosuppressive agents comprise a sphingosine-1-phosphate (S1P) receptor modulator. In some embodiments, one or more immunosuppressive agents comprise a T cell inhibitor. In some embodiments, one or more immunosuppressive agents comprise a tumor necrosis factor alpha (TNF-α) inhibitor. In some embodiments, one or more immunosuppressive agents comprise a protease, e.g., a cysteine protease.

In some embodiments, one or more immunosuppressive agents comprise a steroid. In some embodiments, one or more immunosuppressive agents comprise a corticosteroid. In some embodiments, one or more immunosuppressive agents comprise a glucocorticoid. In some embodiments, one or more immunosuppressive agents comprise dexamethasone. In some embodiments, one or more immunosuppressive agents comprise prednisone. In some embodiments, one or more immunosuppressive agents comprise prednisolone. In some embodiments, one or more immunosuppressive agents comprise methylprednisolone.

In some embodiments, one or more immunosuppressive agents comprise a macrolide. In some embodiments, one or more immunosuppressive agents comprise tacrolimus. In some embodiments, one or more immunosuppressive agents comprise pimecrolimus. In some embodiments, one or more immunosuppressive agents comprise sirolimus (rapamycin). In some embodiments, one or more immunosuppressive agents comprise everolimus. In some embodiments, one or more immunosuppressive agents comprise temsirolimus. In some embodiments, one or more immunosuppressive agents comprise ridaforolimus.

In some embodiments, one or more immunosuppressive agents comprise mycophenolic acid. In some embodiments, one or more immunosuppressive agents comprise mycophenolate. In some embodiments, one or more immunosuppressive agents comprise mycophenolate mofetil (MMF). In some embodiments, one or more immunosuppressive agents comprise mycophenolate sodium (MPS).

In some embodiments, one or more immunosuppressive agents comprise an antibody or antigen binding portion thereof. In some embodiments, an antibody is a monoclonal antibody. In some embodiments, an antibody is a polyclonal antibody. In some embodiments, an antibody or antigen binding portion thereof is an anti-BDCA2 antibody or antigen binding portion thereof. In some embodiments, an antibody or antigen binding portion thereof is an anti-B-lymphocyte stimulator (BLyS) antibody or antigen binding portion thereof, e.g., belimumab. In some embodiments, an antibody or antigen binding portion thereof is an anti-C5 antibody or antigen binding portion thereof, e.g., crovalimab, eculizumab. In some embodiments, the antibody or antigen binding portion thereof is an anti-CD3 antibody or antigen binding portion thereof, e.g., muromonab-CD3. In some embodiments, an antibody is an anti-CD11a antibody or antigen binding portion thereof, e.g., efalizumab. In some embodiments, an antibody or antigen binding portion thereof is an anti-CD20 antibody or antigen binding portion thereof, e.g., ocrelizumab, ofatumumab, rituximab, veltuzumab. In some embodiments, an antibody or antigen binding portion thereof is an anti-CD22 antibody or antigen binding portion thereof, e.g., epratuzumab. In some embodiments, an antibody or antigen binding portion thereof is an anti-CD25 antibody or antigen binding portion thereof, e.g., basiliximab, daclizumab. In some embodiments, an antibody or antigen binding portion thereof is an anti-CD40 antibody or antigen binding portion thereof. In some embodiments, an antibody or antigen binding portion thereof is an anti-CD40L antibody or antigen binding portion thereof. In some embodiments, an antibody or antigen binding portion thereof is an anti-CD52 antibody or antigen binding portion thereof, e.g., alemtuzumab. In some embodiments, an antibody or antigen binding portion thereof is an anti-IL-1B antibody or antigen binding portion thereof, e.g., canakinumab. In some embodiments, an antibody or antigen binding portion thereof is an anti-IL-2 antibody or antigen binding portion thereof. In some embodiments, an antibody or antigen binding portion thereof is an anti-IL-6R antibody or antigen binding portion thereof, e.g., tocilizumab. In some embodiments, an antibody or antigen binding portion thereof is an anti-IL-17A antibody or antigen binding portion thereof, e.g., secukinumab. In some embodiments, an antibody or antigen binding portion thereof is an anti-TNF-α antibody or antigen binding portion thereof, e.g., infliximab, adalimumab, golimumab, certolizumab. In some embodiments, an antibody or antigen binding portion thereof is an anti-lymphocyte antibody or antigen binding portion thereof. In some embodiments, the antibody or antigen binding portion thereof is an anti-T-cell antibody or antigen binding portion thereof. In some embodiments, an antibody is an anti-B-cell antibody or antigen binding portion thereof.

In some embodiments, one or more immunosuppressive agents comprise anti-thymocyte globulin (ATG). In some embodiments, one or more immunosuppressive agents comprise anti-lymphocyte globulin (ALG). In some embodiments, one or more immunosuppressive agents comprise intravenous immune globulin (IVIG).

In some embodiments, one or more immunosuppressive agents comprise a purine analogue. In some embodiments, one or more immunosuppressive agents comprise azathioprine (AZA). In some embodiments, one or more immunosuppressive agents comprise mercaptopurine (6-MP). In some embodiments, one or more immunosuppressive agents comprise a pyrimidine analogue. In some embodiments, one or more immunosuppressive agents comprise fluorouracil (5-FU).

In some embodiments, one or more immunosuppressive agents comprise a fusion protein. In some embodiments, one or more immunosuppressive agents comprise an Fc region of an IgG1, or portion thereof, fused to a CTLA4 extracellular domain, or portion thereof. In some embodiments, one or more immunosuppressive agents comprise abatacept. In some embodiments, one or more immunosuppressive agents comprise belatacept. In some embodiments, one or more immunosuppressive agents comprise an Fc region of an IgG1, or portion thereof, fused to a binding region of transmembrane activator and CAML interactor (TACI), or portion thereof. In some embodiments, one or more immunosuppressive agents comprise atacicept. In some embodiments, one or more immunosuppressive agents comprise an Fc region of an IgG1, or portion thereof, fused to a tumor necrosis factor (TNF) receptor, e.g., TNF receptor 2, or portion thereof. In some embodiments, one or more immunosuppressive agents comprise etanercept. In some embodiments, one or more immunosuppressive agents comprise an Fc region of an IgG1, or portion thereof, fused to a IL-1RAcP extracellular domain, or portion thereof, and/or IL-1R1 extracellular domain, or portion thereof. In some embodiments, one or more immunosuppressive agents comprise rilonacept.

In some embodiments, one or more immunosuppressive agents comprise abrocitinib. In some embodiments, one or more immunosuppressive agents comprise anakinra. In some embodiments, one or more immunosuppressive agents comprise baricitinib. In some embodiments, one or more immunosuppressive agents comprise bortezomib. In some embodiments, one or more immunosuppressive agents comprise cyclophosphamide. In some embodiments, one or more immunosuppressive agents comprise cyclosporine. In some embodiments, one or more immunosuppressive agents comprise fingolimod. In some embodiments, one or more immunosuppressive agents comprise hydroxychloroquine. In some embodiments, one or more immunosuppressive agents comprise a folic acid analogue. In some embodiments, one or more immunosuppressive agents comprise imlifidase. In some embodiments, one or more immunosuppressive agents comprise leflunomide. In some embodiments, one or more immunosuppressive agents comprise methotrexate. In some embodiments, one or more immunosuppressive agents comprise ruxolitinib. In some embodiments, one or more immunosuppressive agents comprise tofacitinib. In some embodiments, one or more immunosuppressive agents comprise upadacitinib.

In some embodiments, one or more immunosuppressive agents comprise a combination of any immunosuppressive agents described herein (e.g., one, two, three, four, five, six, seven, eight, nine, or more immunosuppressive agents described herein).

As described herein, one or more immunosuppressive agents can be administered to a subject. In some embodiments, two or more immunosuppressive agents are administered to a subject. In some embodiments, three or more immunosuppressive agents are administered to a subject. In some embodiments, four or more immunosuppressive agents are administered to a subject. In some embodiments, five or more immunosuppressive agents are administered to a subject.

As described herein, one or more immunosuppressive agents can be administered to a subject as part of an immunosuppressive regimen. In some embodiments, two or more immunosuppressive agents described herein are administered to a subject as part of an immunosuppressive regimen. In some embodiments, three or more immunosuppressive agents described herein are administered to a subject as part of an immunosuppressive regimen. In some embodiments, four or more immunosuppressive agents described herein are administered to a subject as part of an immunosuppressive regimen. In some embodiments, five or more immunosuppressive agents described herein are administered to a subject as part of an immunosuppressive regimen. In some embodiments, an immunosuppressive regimen described herein is administered to a subject.

In some embodiments, an immunosuppressive regimen comprises one or more immunosuppressive agents described herein. In some embodiments, an immunosuppressive regimen comprises two or more immunosuppressive agents described herein. In some embodiments, an immunosuppressive regimen comprises three or more immunosuppressive agents described herein. In some embodiments, an immunosuppressive regimen comprises four or more immunosuppressive agents described herein. In some embodiments, an immunosuppressive regimen comprises five or more immunosuppressive agents described herein. In some embodiments, an immunosuppressive regimen comprises a therapeutically effective amount of one or more immunosuppressive agents described herein. In some embodiments, an immunosuppressive regimen comprises a therapeutically effective amount of two or more immunosuppressive agents described herein. In some embodiments, an immunosuppressive regimen comprises a therapeutically effective amount of three or more immunosuppressive agents described herein. In some embodiments, an immunosuppressive regimen comprises a therapeutically effective amount of four or more immunosuppressive agents described herein. In some embodiments, an immunosuppressive regimen comprises a therapeutically effective amount of five or more immunosuppressive agents described herein.

In some embodiments, an immunosuppressive regimen comprises dexamethasone. In some embodiments, an immunosuppressive regimen comprises dexamethasone and one or more additional immunosuppressive agents described herein. In some embodiments, an immunosuppressive regimen comprises dexamethasone and a calcincurin inhibitor. In some embodiments, an immunosuppressive regimen comprises dexamethasone, a calcineurin inhibitor, and one or more additional immunosuppressive agents described herein. In some embodiments, an immunosuppressive regimen comprises dexamethasone and a macrolide. In some embodiments, an immunosuppressive regimen comprises dexamethasone, a macrolide, and one or more additional immunosuppressive agents described herein. In some embodiments, an immunosuppressive regimen comprises dexamethasone and tacrolimus. In some embodiments, an immunosuppressive regimen comprises dexamethasone, tacrolimus, and one or more additional immunosuppressive agents described herein. In some embodiments, an immunosuppressive regimen comprises dexamethasone and tofacitinib. In some embodiments, an immunosuppressive regimen comprises dexamethasone, tofacitinib, and one or more additional immunosuppressive agents described herein. In some embodiments, an immunosuppressive regimen comprises dexamethasone, tacrolimus, and mycophenolate mofetil (MMF). In some embodiments, an immunosuppressive regimen comprises dexamethasone, tacrolimus, MMF, and one or more additional immunosuppressive agents described herein. In some embodiments, an immunosuppressive regimen comprises dexamethasone, tacrolimus, and tofacitinib. In some embodiments, an immunosuppressive regimen comprises dexamethasone, tacrolimus, tofacitinib, and one or more additional immunosuppressive agents described herein.

### Methods of Treatment

As appreciated by those skilled in the art, rAAVs, immunosuppressive regimens, and immunosuppressive agents are useful for many purposes. In some embodiments, provided technologies (e.g., rAAVs, immunosuppressive regimens, immunosuppressive agents, and methods thereof) are useful for administering to and/or treating subjects.

The administration of viral vectors, e.g., rAAVs, have reportedly been associated with toxicity in various organs, tissues, and/or cells of subjects who received said viral vectors. For example, administration of rAAVs has been reportedly associated with hepatotoxicity. In addition, administration of rAAVs, particularly those targeted to the central nervous system (CNS), has been associated with dorsal root ganglion (DRG) toxicity in various models including mice, rats, pigs, and non-human primates. See, e.g., Bolt et al., J Toxicol Sci. 2021; 46 (2): 57-68; Fader et al., Mol Ther Methods Clin Dev. 2022 Jun. 9; 25:264-277; Hordeaux et al., Hum Gene Ther. 2020 August; 31 (15-16): 808-818; Hordeaux et al., Mol Ther Methods Clin Dev. 2018 Jul. 14; 10:68-78; Hinderer et al., Hum Gene Ther. 2018 March; 29 (3): 285-298; Palazzi et al., Hum Gene Ther. 2022 February;33 (3-4): 175-187; Tukov et al., Hum Gene Ther. 2022 July; 33 (13-14): 740-756, each of which is hereby incorporated by reference in its entirety. In these reports, lesions (e.g., axonal/nerve fiber degeneration, neuronal cell body degeneration/necrosis, increased immune cell infiltrates, and/or gliosis) have been observed in the DRG and associated spinal cord segments and nerves following administration of a rAAV. Notably, this toxicity has been observed with a variety of rAAVs, including those with various different capsids and nucleic acids (e.g., various different promoters, various different transgenes). The causative factors of this DRG toxicity are not fully understood; however, reports have indicated that transgene overexpression may play an important role. See, e.g., Buss et al., Mol Ther Methods Clin Dev. 2022 Feb. 1; 24:342-354, hereby incorporated by reference in its entirety.

In an attempt to address toxicity observed with administration of viral vectors, e.g., rAAVs, various approaches have been attempted. For example, rAAVs may be designed with various nucleic acid sequences (e.g., promoters, enhancers, or other 5′- or 3′-untranslated region elements) to alter transgene expression levels and/or administered via variable routes and dosages. Yet, such methodology may not be applicable or capable of reducing toxicity as desired. Attenuation of transgene expression (by, e.g., inclusion of sequences recognized by particular miRNAs) in specific tissues has been attempted to lower transgene expression in toxicity-susceptible tissues. See, e.g., Hordeaux et al., Hum Gene Ther. 2020 August; 31 (15-16): 808-818, hereby incorporated by reference in its entirety. Others have implemented immunosuppressive regimens as an attempt to reduce or prevent potentially damaging immune system reactions to the rAAVs. See, e.g., Prasad et al., Hum Gene Ther. 2022 December; 33 (23-24): 1228-1245, hereby incorporated by reference in its entirety. However, reduction of rAAV-mediated toxicity, particularly DRG toxicity, has not yet been effectively addressed. The present disclosure has identified, amongst other things, potential causal factors in DRG toxicity and immunosuppressive regimens, immunosuppressive agents, and methods thereof that may reduce DRG toxicity. In some embodiments, provided technologies (e.g., rAAVs, immunosuppressive regimens, immunosuppressive agents, and methods thereof) reduce toxicity in a subject, e.g., DRG toxicity. In some embodiments, provided technologies reduce severity and/or incidence of lesions in one or more DRG. In some embodiments, provided technologies reduce neural degeneration in one or more DRG. In some embodiments, provided technologies reduce neuron cell body degeneration and/or necrosis in one or more DRG. In some embodiments, provided technologies reduce nerve fiber degeneration in one or more DRG. In some embodiments, provided technologies reduce axonal degeneration in one or more DRG. In some embodiments, provided technologies reduce mononuclear cell infiltration in one or more DRG. In some embodiments, provided technologies reduce severity and/or incidence of lesions in spinal cord tissue. In some embodiments, provided technologies reduce neural degeneration in spinal cord tissue. In some embodiments, provided technologies reduce neuron cell body degeneration and/or necrosis in spinal cord tissue. In some embodiments, provided technologies reduce nerve fiber degeneration in spinal cord tissue. In some embodiments, provided technologies reduce axonal degeneration in spinal cord tissue. In some embodiments, provided technologies reduce mononuclear cell infiltration in spinal cord tissue.

In some embodiments, the present disclosure provides methods of administering to a subject a rAAV (e.g., a therapeutically effective amount of a rAAV) and an immunosuppressive regimen as provided herein. In some embodiments, the present disclosure provides methods of treating a subject, comprising administering a rAAV and an immunosuppressive regimen as provided herein to such subject.

In some embodiments, the present disclosure provides methods of reducing DRG toxicity in a subject, comprising administering a rAAV and an immunosuppressive regimen as provided herein to such subject. In some embodiments, the present disclosure provides methods of reducing lesion severity and/or incidence in one or more DRG in a subject, comprising administering a rAAV and an immunosuppressive regimen as provided herein to such subject. In some embodiments, the present disclosure provides methods of reducing neural degeneration in one or more DRG in a subject, comprising administering a rAAV and an immunosuppressive regimen as provided herein to such subject. In some embodiments, the present disclosure provides methods of reducing neuronal cell body degeneration and/or necrosis in one or more DRG in a subject, comprising administering a rAAV and an immunosuppressive regimen as provided herein to such subject. In some embodiments, the present disclosure provides methods of reducing nerve fiber degeneration in one or more DRG in a subject, comprising administering a rAAV and an immunosuppressive regimen as provided herein to such subject. In some embodiments, the present disclosure provides methods of reducing axonal degeneration in one or more DRG in a subject, comprising administering a rAAV and an immunosuppressive regimen as provided herein to such subject. In some embodiments, the present disclosure provides methods of reducing mononuclear cell infiltration in one or more DRG in a subject, comprising administering a rAAV and an immunosuppressive regimen as provided herein to such subject.

In some embodiments, the present disclosure provides methods of reducing spinal cord toxicity in a subject, comprising administering a rAAV and an immunosuppressive regimen as provided herein to such subject. In some embodiments, the present disclosure provides methods of reducing lesion severity and/or incidence in spinal cord tissue in a subject, comprising administering a rAAV and an immunosuppressive regimen as provided herein to such subject. In some embodiments, the present disclosure provides methods of reducing neural degeneration in spinal cord tissue in a subject, comprising administering a rAAV and an immunosuppressive regimen as provided herein to such subject. In some embodiments, the present disclosure provides methods of reducing neuronal cell body degeneration and/or necrosis in spinal cord tissue in a subject, comprising administering a rAAV and an immunosuppressive regimen as provided herein to such subject. In some embodiments, the present disclosure provides methods of reducing nerve fiber degeneration in spinal cord tissue in a subject, comprising administering a rAAV and an immunosuppressive regimen as provided herein to such subject. In some embodiments, the present disclosure provides methods of reducing axonal degeneration in spinal cord tissue in a subject, comprising administering a rAAV and an immunosuppressive regimen as provided herein to such subject. In some embodiments, the present disclosure provides methods of reducing mononuclear cell infiltration in spinal cord tissue in a subject, comprising administering a rAAV and an immunosuppressive regimen as provided herein to such subject.

In some embodiments, the present disclosure provides methods of treating a subject, comprising administering a rAAV and an immunosuppressive regimen comprising one or more immunosuppressive agents (e.g., immunosuppressive agents described herein) to such subject. In some embodiments, a method of treating a subject comprises administering a rAAV and an immunosuppressive regimen comprising dexamethasone and one or more additional immunosuppressive agents (e.g., immunosuppressive agents described herein) to such subject. In some embodiments, a method of treating a subject comprises administering a rAAV and an immunosuppressive regimen comprising dexamethasone and a calcineurin inhibitor to such subject. In some embodiments, a method of treating a subject comprises administering a rAAV and an immunosuppressive regimen comprising dexamethasone, a calcineurin inhibitor, and one or more additional immunosuppressive agents (e.g., immunosuppressive agents described herein) to such subject. In some embodiments, a method of treating a subject comprises administering a rAAV and an immunosuppressive regimen comprising dexamethasone, a calcineurin inhibitor, and MMF to such subject. In some embodiments, a method of treating a subject comprises administering a rAAV and an immunosuppressive regimen comprising dexamethasone, a calcineurin inhibitor, and tofacitinib to such subject.

In some embodiments, the present disclosure provides methods of reducing DRG toxicity in a subject being administered a rAAV, comprising administering such rAAV and an immunosuppressive regimen (comprising, e.g., one or more immunosuppressive regimens described herein) to such subject. In some embodiments, a method of reducing DRG toxicity in a subject being administered a rAAV, comprises administering such rAAV and an immunosuppressive regimen comprising dexamethasone and one or more additional immunosuppressive agents to such subject. In some embodiments, a method of reducing DRG toxicity in a subject being administered a rAAV comprises administering such rAAV and an immunosuppressive regimen comprising dexamethasone and a calcineurin inhibitor to such subject. In some embodiments, a method of reducing DRG toxicity in a subject being administered a rAAV comprises administering such rAAV and an immunosuppressive regimen comprising dexamethasone, a calcineurin inhibitor, and one or more additional immunosuppressive agents to such subject. In some embodiments, a method of reducing DRG toxicity in a subject being administered a rAAV comprises administering such rAAV and an immunosuppressive regimen comprising dexamethasone, a calcineurin inhibitor, and MMF to such subject. In some embodiments, a method of reducing DRG toxicity in a subject being administered a rAAV comprises administering such rAAV and an immunosuppressive regimen comprising dexamethasone, a calcineurin inhibitor, and tofacitinib to such subject.

In some embodiments, the present disclosure provides methods of reducing lesion severity and/or incidence in one or more DRG in a subject being administered a rAAV, comprising administering such rAAV and an immunosuppressive regimen (e.g., an immunosuppressive regimen described herein) to the subject. In some embodiments, a method of reducing lesion severity and/or incidence in one or more DRG in a subject being administered a rAAV, comprises administering such rAAV and an immunosuppressive regimen comprising dexamethasone and one or more additional immunosuppressive agents to such subject. In some embodiments, a method of reducing lesion severity and/or incidence in one or more DRG in a subject being administered a rAAV comprises administering such rAAV and an immunosuppressive regimen comprising dexamethasone and a calcineurin inhibitor to such subject. In some embodiments, a method of reducing lesion severity and/or incidence in one or more DRG in a subject being administered a rAAV comprises administering such rAAV and an immunosuppressive regimen comprising dexamethasone, a calcineurin inhibitor, and one or more additional immunosuppressive agents to such subject. In some embodiments, a method of reducing lesion severity and/or incidence in one or more DRG in a subject being administered a rAAV comprises administering such rAAV and an immunosuppressive regimen comprising dexamethasone, a calcineurin inhibitor, and MMF to such subject. In some embodiments, a method of reducing lesion severity and/or incidence in one or more DRG in a subject being administered a rAAV comprises administering such rAAV and an immunosuppressive regimen comprising dexamethasone, a calcineurin inhibitor, and tofacitinib to such subject.

In some embodiments, the present disclosure provides methods of reducing axonal degeneration in one or more DRG in a subject being administered a rAAV, comprising administering a rAAV and an immunosuppressive regimen (e.g., an immunosuppressive regimen described herein, e.g., comprising one or more immunosuppressive agents described herein) to such subject. In some embodiments, a method of reducing axonal degeneration in one or more DRG in a subject being administered a rAAV comprises administering a rAAV and an immunosuppressive regimen comprising dexamethasone and one or more additional immunosuppressive agents to such subject. In some embodiments, a method of reducing axonal degeneration in one or more DRG in a subject being administered a rAAV comprises administering such rAAV and an immunosuppressive regimen comprising dexamethasone and a calcineurin inhibitor to such subject. In some embodiments, a method of reducing axonal degeneration in one or more DRG in a subject being administered a rAAV comprises administering such rAAV and an immunosuppressive regimen comprising dexamethasone, a calcincurin inhibitor, and one or more additional immunosuppressive agents to such subject. In some embodiments, a method of reducing axonal degeneration in one or more DRG in a subject being administered a rAAV comprises administering such rAAV and an immunosuppressive regimen comprising dexamethasone, a calcineurin inhibitor, and MMF to such subject. In some embodiments, a method of reducing axonal degeneration in one or more DRG in a subject being administered a rAAV comprises administering such rAAV and an immunosuppressive regimen comprising dexamethasone, a calcineurin inhibitor, and tofacitinib to such subject.

In some embodiments, DRG toxicity is reduced relative to a reference condition. In some embodiments, DRG toxicity is reduced relative to administration of arAAV without an immunosuppressive regimen described herein. In some embodiments, lesion severity and/or incidence in one or more DRG is reduced relative to a reference condition. In some embodiments, lesion severity and/or incidence in one or more DRG is reduced relative to administration of a rAAV without an immunosuppressive regimen described herein. In some embodiments, axonal degeneration in one or more DRG is reduced relative to a reference condition. In some embodiments, axonal degeneration in one or more DRG is reduced relative to administration of a rAAV without an immunosuppressive regimen described herein.

In some embodiments, a reference condition comprises administration of the rAAV without an immunosuppressive regimen described herein. In some embodiments, a reference condition comprises administration of a rAAV with an alternative immunosuppressive regimen lacking one or more immunosuppressive agents as compared to an immunosuppressive regimen described herein.

In some embodiments, a subject is a mammal. In some embodiments, a subject is a mouse. In some embodiments, a subject is a rat. In some embodiments, a subject is a dog. In some embodiments, a subject is a pig. In some embodiments, a subject is a non-human primate, e.g., a juvenile non-human primate. In some embodiments, a subject is a human. In some embodiments, a subject is an adult, e.g., a human adult. In some embodiments, a subject is a pediatric subject, e.g., human child.

### Administration and Dosing

As described herein, compositions comprising rAAVs and/or compositions comprising one or more immunosuppressive agents can be administered to a subject. In some embodiments, rAAVs and/or one or more immunosuppressive agents are administered by a suitable route as known in the art. In some embodiments, rAAVs are administered by an intravenous, intrathecal, intracerebroventricular, intracisternal, intramuscular, intraparenchymal, intracranial, intraocular, intra-articular, intranasal, intraosseous, intra-alveolar, intra-arterial, intraperitoneal, oral, subcutaneous, sublingual, or submucosal route. In some embodiments, one or more immunosuppressive agents are administered by an intravenous, intrathecal, intracerebroventricular, intracisternal, intramuscular, intraparenchymal, intracranial, intraocular, intra-articular, intranasal, intraosseous, intra-alveolar, intra-arterial, intraperitoneal, oral, subcutaneous, sublingual, or submucosal route.

Various routes of administration for AAVs (e.g., rAAVs) are known in the art. In some embodiments, a rAAV is administered systemically. In some embodiments, a rAAV is administered intravenously, intrathecally, intracerebroventricularly, intracisternally, intramuscularly, intraparenchymally, intracranially, intraocularly, intraarticularly, intranasally, intrathecally, and/or subcutaneously. In some embodiments, a rAAV is administered intravenously. In some embodiments, a rAAV is administered intrathecally. In some embodiments, a rAAV is administered intracerebroventricularly. In some embodiments, a rAAV is administered intracisternally. In some embodiments, a rAAV is administered intramuscularly. In some embodiments, a rAAV is administered intraparenchymally. In some embodiments, a rAAV is administered intracranially. In some embodiments, a rAAV is administered intraocularly. In some embodiments, a rAAV is administered intraarticularly. In some embodiments, a rAAV is administered intranasally. In some embodiments, a rAAV is administered intrathecally. In some embodiments, a rAAV is administered subcutaneously.

In some embodiments, a rAAV is administered at a dose of between about 10⁷ to about 10¹⁸ viral genomes (vg). In some embodiments, a rAAV is administered at a dose of about 10⁷ vg, about 10⁸ vg, about 10⁹ vg, about 10¹⁰ vg, about 10¹¹ vg, about 10¹² vg, about 10¹³ vg, about 10¹⁴ vg, about 10¹⁵ vg, about 10¹⁶ vg, about 10¹⁷ vg, or about 10¹⁸ vg.

In some embodiments, one or more immunosuppressants is administered intravenously, intrathecally, intracerebroventricularly, intracisternally, intramuscularly, intraparenchymally, intracranially, intraocularly, intraosseously, intraarticularly, intranasally, subcutaneously, and/or orally. In some embodiments, one or more immunosuppressants is administered intravenously. In some embodiments, one or more immunosuppressants is administered intrathecally. In some embodiments, one or more immunosuppressants are administered intracerebroventricularly. In some embodiments, one or more immunosuppressants are administered intracisternally. In some embodiments, one or more immunosuppressants are administered intramuscularly. In some embodiments, one or more immunosuppressants are administered intraparenchymally. In some embodiments, one or more immunosuppressants are administered intracranially. In some embodiments, one or more immunosuppressants are administered intraocularly. In some embodiments, one or more immunosuppressants is administered intraosseously. In some embodiments, one or more immunosuppressants are administered intraarticularly. In some embodiments, one or more immunosuppressants arc administered intranasally. In some embodiments, one or more immunosuppressants is administered subcutaneously. In some embodiments, one or more immunosuppressants is administered orally.

In some embodiments, one or more immunosuppressants is administered every other day, once a day, twice a day, three times a day, or four times a day. In some embodiments, one or more immunosuppressants is administered every other day. In some embodiments, one or more immunosuppressants is administered once a day. In some embodiments, one or more immunosuppressants is administered twice a day. In some embodiments, one or more immunosuppressants is administered three times a day. In some embodiments, one or more immunosuppressants is administered four times a day.

In some embodiments, dexamethasone is administered intraosseously, intrathecally, intravenously, and/or orally. In some embodiments, dexamethasone is administered intraosseously. In some embodiments, dexamethasone is administered intrathecally. In some embodiments, dexamethasone is administered intravenously. In some embodiments, dexamethasone is administered orally.

In some embodiments, dexamethasone is administered every other day, once a day, twice a day, three times a day, or four times a day. In some embodiments, dexamethasone is administered every other day. In some embodiments, dexamethasone is administered once a day. In some embodiments, dexamethasone is administered twice a day. In some embodiments, dexamethasone is administered three times a day. In some embodiments, dexamethasone is administered four times a day.

In some embodiments, dexamethasone is administered at a dose of between about 0.01 mg/kg to about 10 mg/kg. In some embodiments, dexamethasone is administered at a dose of between about 0.1 mg/kg to about 10 mg/kg. In some embodiments, dexamethasone is administered at a dose of between about 0.1 mg/kg to about 5 mg/kg. In some embodiments, dexamethasone is administered at a dose of between about 0.1 mg/kg to about 2.5 mg/kg. In some embodiments, dexamethasone is administered at a dose of between about 0.25 mg/kg to about 2.5 mg/kg. In some embodiments, dexamethasone is administered at a dose of between about 0.1 mg/kg to about 1 mg/kg. In some embodiments, dexamethasone is administered at a dose of between about 0.5 mg/kg to about 1 mg/kg. In some embodiments, dexamethasone is administered at a dose of about 0.1 mg/kg. In some embodiments, dexamethasone is administered at a dose of about 0.15 mg/kg. In some embodiments, dexamethasone is administered at a dose of about 0.2 mg/kg. In some embodiments, dexamethasone is administered at a dose of about 0.25 mg/kg. In some embodiments, dexamethasone is administered at a dose of about 0.3 mg/kg. In some embodiments, dexamethasone is administered at a dose of about 0.35 mg/kg. In some embodiments, dexamethasone is administered at a dose of about 0.4 mg/kg. In some embodiments, dexamethasone is administered at a dose of about 0.45 mg/kg. In some embodiments, dexamethasone is administered at a dose of about 0.5 mg/kg. In some embodiments, dexamethasone is administered at a dose of about 0.55 mg/kg. In some embodiments, dexamethasone is administered at a dose of about 0.6 mg/kg. In some embodiments, dexamethasone is administered at a dose of about 0.65 mg/kg. In some embodiments, dexamethasone is administered at a dose of about 0.7 mg/kg. In some embodiments, dexamethasone is administered at a dose of about 0.75 mg/kg. In some embodiments, dexamethasone is administered at a dose of about 0.8 mg/kg. In some embodiments, dexamethasone is administered at a dose of about 0.85 mg/kg. In some embodiments, dexamethasone is administered at a dose of about 0.9 mg/kg. In some embodiments, dexamethasone is administered at a dose of about 0.95 mg/kg. In some embodiments, dexamethasone is administered at a dose of about 1 mg/kg.

In some embodiments, dexamethasone is administered on each of 1, 2, 3, 4, 5, 6, 7, 8, 9, 10, or more days prior to administration of a rAAV. In some embodiments, dexamethasone is administered on a same day as administration of a rAAV. In some embodiments, dexamethasone is administered on each day following administration of a rAAV for about 1 week, 2 weeks, 3 weeks, 4 weeks, 1 month, 2 months, 3 months, 4 months, 5 months, 6 months, or longer. In some embodiments, dexamethasone is administered every other day for 1, 2, 3, 4, 5, 6, 7, 8, 9, 10, or more days prior to administration of a rAAV. In some embodiments, dexamethasone is administered on a same day as administration of a rAAV. In some embodiments, dexamethasone is administered on every other day following administration of a rAAV for about 1 week, 2 weeks, 3 weeks, 4 weeks, 1 month, 2 months, 3 months, 4 months, 5 months, 6 months, or longer.

In some embodiments, a calcineurin inhibitor is administered intravenously and/or orally. In some embodiments, a calcineurin inhibitor is administered intravenously. In some embodiments, a calcineurin inhibitor is administered orally. In some embodiments, a macrolide is administered intravenously and/or orally. In some embodiments, a macrolide is administered intravenously. In some embodiments, a macrolide is administered orally. In some embodiments, tacrolimus is administered intravenously and/or orally. In some embodiments, tacrolimus is administered intravenously. In some embodiments, tacrolimus is administered orally.

In some embodiments, a calcineurin inhibitor is administered every other day, once a day, twice a day, three times a day, or four times a day. In some embodiments, a calcineurin inhibitor is administered every other day. In some embodiments, a calcineurin inhibitor is administered once a day. In some embodiments, a calcineurin inhibitor is administered twice a day. In some embodiments, a calcineurin inhibitor is administered three times a day. In some embodiments, a calcineurin inhibitor is administered four times a day. In some embodiments, a macrolide is administered every other day, once a day, twice a day, three times a day, or four times a day. In some embodiments, a macrolide is administered every other day. In some embodiments, a macrolide is administered once a day. In some embodiments, a macrolide is administered twice a day. In some embodiments, a macrolide is administered three times a day. In some embodiments, a macrolide is administered four times a day. In some embodiments, tacrolimus is administered every other day, once a day, twice a day, three times a day, or four times a day. In some embodiments, tacrolimus is administered every other day. In some embodiments, tacrolimus is administered once a day. In some embodiments, tacrolimus is administered twice a day. In some embodiments, tacrolimus is administered three times a day. In some embodiments, tacrolimus is administered four times a day.

In some embodiments, a calcineurin inhibitor is administered at a dose of between about 0.01 mg/kg to about 10 mg/kg. In some embodiments, a calcineurin inhibitor is administered at a dose of between about 0.1 mg/kg to about 10 mg/kg. In some embodiments, a calcineurin inhibitor is administered at a dose of between about 0.1 mg/kg to about 5 mg/kg. In some embodiments, a calcineurin inhibitor is administered at a dose of between about 0.1 mg/kg to about 2.5 mg/kg. In some embodiments, a calcineurin inhibitor is administered at a dose of between about 0.25 mg/kg to about 2.5 mg/kg. In some embodiments, a calcineurin inhibitor is administered at a dose of between about 0.1 mg/kg to about 1 mg/kg. In some embodiments, a calcineurin inhibitor is administered at a dose of between about 0.5 mg/kg to about 1 mg/kg. In some embodiments, a calcineurin inhibitor is administered at a dose of about 0.1 mg/kg. In some embodiments, a calcineurin inhibitor is administered at a dose of about 0.15 mg/kg. In some embodiments, a calcineurin inhibitor is administered at a dose of about 0.2 mg/kg. In some embodiments, a calcineurin inhibitor is administered at a dose of about 0.25 mg/kg. In some embodiments, a calcineurin inhibitor is administered at a dose of about 0.3 mg/kg. In some embodiments, a calcineurin inhibitor is administered at a dose of about 0.35 mg/kg. In some embodiments, a calcineurin inhibitor is administered at a dose of about 0.4 mg/kg. In some embodiments, a calcineurin inhibitor is administered at a dose of about 0.45 mg/kg. In some embodiments, a calcineurin inhibitor is administered at a dose of about 0.5 mg/kg. In some embodiments, a calcineurin inhibitor is administered at a dose of about 0.55 mg/kg. In some embodiments, a calcineurin inhibitor is administered at a dose of about 0.6 mg/kg. In some embodiments, a calcineurin inhibitor is administered at a dose of about 0.65 mg/kg. In some embodiments, a calcineurin inhibitor is administered at a dose of about 0.7 mg/kg. In some embodiments, a calcineurin inhibitor is administered at a dose of about 0.75 mg/kg. In some embodiments, a calcineurin inhibitor is administered at a dose of about 0.8 mg/kg. In some embodiments, a calcineurin inhibitor is administered at a dose of about 0.85 mg/kg. In some embodiments, a calcineurin inhibitor is administered at a dose of about 0.9 mg/kg. In some embodiments, a calcineurin inhibitor is administered at a dose of about 0.95 mg/kg. In some embodiments, a calcineurin inhibitor is administered at a dose of about 1 mg/kg. In some embodiments, a calcineurin inhibitor is administered at a dose of about 1.1 mg/kg. In some embodiments, a calcincurin inhibitor is administered at a dose of about 1.2 mg/kg. In some embodiments, a calcineurin inhibitor is administered at a dose of about 1.3 mg/kg. In some embodiments, a calcineurin inhibitor is administered at a dose of about 1.4 mg/kg. In some embodiments, a calcineurin inhibitor is administered at a dose of about 1.5 mg/kg.

In some embodiments, tacrolimus is administered at a dose of between about 0.01 mg/kg to about 10 mg/kg. In some embodiments, tacrolimus is administered at a dose of between about 0.1 mg/kg to about 10 mg/kg. In some embodiments, tacrolimus is administered at a dose of between about 0.1 mg/kg to about 5 mg/kg. In some embodiments, tacrolimus is administered at a dose of between about 0.1 mg/kg to about 2.5 mg/kg. In some embodiments, tacrolimus is administered at a dose of between about 0.25 mg/kg to about 2.5 mg/kg. In some embodiments, tacrolimus is administered at a dose of between about 0.1 mg/kg to about 1 mg/kg. In some embodiments, tacrolimus is administered at a dose of between about 0.5 mg/kg to about 1 mg/kg. In some embodiments, tacrolimus is administered at a dose of about 0.1 mg/kg. In some embodiments, tacrolimus is administered at a dose of about 0.15 mg/kg. In some embodiments, tacrolimus is administered at a dose of about 0.2 mg/kg. In some embodiments, tacrolimus is administered at a dose of about 0.25 mg/kg. In some embodiments, tacrolimus is administered at a dose of about 0.3 mg/kg. In some embodiments, tacrolimus is administered at a dose of about 0.35 mg/kg. In some embodiments, tacrolimus is administered at a dose of about 0.4 mg/kg. In some embodiments, tacrolimus is administered at a dose of about 0.45 mg/kg. In some embodiments, tacrolimus is administered at a dose of about 0.5 mg/kg. In some embodiments, tacrolimus is administered at a dose of about 0.55 mg/kg. In some embodiments, tacrolimus is administered at a dose of about 0.6 mg/kg. In some embodiments, tacrolimus is administered at a dose of about 0.65 mg/kg. In some embodiments, tacrolimus is administered at a dose of about 0.7 mg/kg. In some embodiments, tacrolimus is administered at a dose of about 0.75 mg/kg. In some embodiments, tacrolimus is administered at a dose of about 0.8 mg/kg. In some embodiments, tacrolimus is administered at a dose of about 0.85 mg/kg. In some embodiments, tacrolimus is administered at a dose of about 0.9 mg/kg. In some embodiments, tacrolimus is administered at a dose of about 0.95 mg/kg. In some embodiments, tacrolimus is administered at a dose of about 1 mg/kg. In some embodiments, tacrolimus is administered at a dose of about 1.1 mg/kg. In some embodiments, tacrolimus is administered at a dose of about 1.2 mg/kg. In some embodiments, tacrolimus is administered at a dose of about 1.3 mg/kg. In some embodiments, tacrolimus is administered at a dose of about 1.4 mg/kg. In some embodiments, tacrolimus is administered at a dose of about 1.5 mg/kg.

In some embodiments, a calcineurin inhibitor is administered on each of 1, 2, 3, 4, 5, 6, 7, 8, 9, 10, or more days prior to administration of a rAAV. In some embodiments, a calcineurin inhibitor is administered on a same day as administration of arAAV. In some embodiments, a calcincurin inhibitor is administered on each day following administration of a rAAV for about 1 week, 2 weeks, 3 weeks, 4 weeks, 1 month, 2 months, 3 months, 4 months, 5 months, 6 months, or longer. In some embodiments, a calcineurin inhibitor is administered every other day for 1, 2, 3, 4, 5, 6, 7, 8, 9, 10, or more days prior to administration of arAAV. In some embodiments, a calcineurin inhibitor is administered on a same day as administration of a rAAV. In some embodiments, a calcineurin inhibitor is administered on every other day following administration of a rAAV for about 1 week, 2 weeks, 3 weeks, 4 weeks, 1 month, 2 months, 3 months, 4 months, 5 months, 6 months, or longer. In some embodiments, a macrolide is administered on each of 1, 2, 3, 4, 5, 6, 7, 8, 9, 10, or more days prior to administration of arAAV. In some embodiments, a macrolide is administered on a same day as administration of a rAAV. In some embodiments, a macrolide is administered on each day following administration of arAAV for about 1 week, 2 weeks, 3 weeks, 4 weeks, 1 month, 2 months, 3 months, 4 months, 5 months, 6 months, or longer. In some embodiments, a macrolide is administered every other day for 1, 2, 3, 4, 5, 6, 7, 8, 9, 10, or more days prior to administration of a rAAV. In some embodiments, a macrolide is administered on a same day as administration of a rAAV. In some embodiments, a macrolide is administered on every other day following administration of a rAAV for about 1 week, 2 weeks, 3 weeks, 4 weeks, 1 month, 2 months, 3 months, 4 months, 5 months, 6 months, or longer.

In some embodiments, tacrolimus is administered on each of 1, 2, 3, 4, 5, 6, 7, 8, 9, 10, or more days prior to administration of a rAAV. In some embodiments, tacrolimus is administered on a same day as administration of a rAAV. In some embodiments, tacrolimus is administered on each day following administration of a rAAV for about 1 week, 2 weeks, 3 weeks, 4 weeks, 1 month, 2 months, 3 months, 4 months, 5 months, 6 months, or longer. In some embodiments, tacrolimus is administered every other day for 1, 2, 3, 4, 5, 6, 7, 8, 9, 10, or more days prior to administration of a rAAV. In some embodiments, tacrolimus is administered on a same day as administration of a rAAV. In some embodiments, tacrolimus is administered on every other day following administration of a rAAV for about 1 week, 2 weeks, 3 weeks, 4 weeks, 1 month, 2 months, 3 months, 4 months, 5 months, 6 months, or longer.

In some embodiments, mycophenolate mofetil (MMF) is administered intravenously and/or orally. In some embodiments, MMF is administered intravenously. In some embodiments, MMF is administered orally.

In some embodiments, mycophenolate mofetil (MMF) is administered every other day, once a day, twice a day, three times a day, or four times a day. In some embodiments, MMF is administered every other day. In some embodiments, MMF is administered once a day. In some embodiments, MMF is administered twice a day. In some embodiments, MMF is administered three times a day. In some embodiments, MMF is administered four times a day.

In some embodiments, mycophenolate mofetil (MMF) is administered at a dose of between about 0.1 mg/kg to about 200 mg/kg. In some embodiments, MMF is administered at a dose of between about 1 mg/kg to about 100 mg/kg. In some embodiments, MMF is administered at a dose of between about 10 mg/kg to about 75 mg/kg. In some embodiments, MMF is administered at a dose of between about 5 mg/kg to about 50 mg/kg. In some embodiments, MMF is administered at a dose of between about 10 mg/kg to about 50 mg/kg. In some embodiments, MMF is administered at a dose of between about 25 mg/kg to about 50 mg/kg. In some embodiments, MMF is administered at a dose of about 5 mg/kg. In some embodiments, MMF is administered at a dose of about 10 mg/kg. In some embodiments, MMF is administered at a dose of about 15 mg/kg. In some embodiments, MMF is administered at a dose of about 20 mg/kg. In some embodiments, MMF is administered at a dose of about 25 mg/kg. In some embodiments, MMF is administered at a dose of about 30 mg/kg. In some embodiments, MMF is administered at a dose of about 35 mg/kg. In some embodiments, MMF is administered at a dose of about 40 mg/kg. In some embodiments, MMF is administered at a dose of about 45 mg/kg. In some embodiments, MMF is administered at a dose of about 50 mg/kg.

In some embodiments, MMF is administered at a dose of between about 0.1 g to about 10 g. In some embodiments, MMF is administered at a dose of between about 0.1 g to about 5 g. In some embodiments, MMF is administered at a dose of between about 0.5 g to about 5 g. In some embodiments, MMF is administered at a dose of between about 1 g to about 5 g. In some embodiments, MMF is administered at a dose of between about 0.1 g to about 2.5 g. In some embodiments, MMF is administered at a dose of between about 0.5 g to about 2.5 g. In some embodiments, MMF is administered at a dose of between about 1 g to about 2.5 g. In some embodiments, MMF is administered at a dose of about 0.5 g. In some embodiments, MMF is administered at a dose of about 0.6 g. In some embodiments, MMF is administered at a dose of about 0.7 g. In some embodiments, MMF is administered at a dose of about 0.8 g. In some embodiments, MMF is administered at a dose of about 0.9 g. In some embodiments, MMF is administered at a dose of about 1 g. In some embodiments, MMF is administered at a dose of about 1.1 g. In some embodiments, MMF is administered at a dose of about 1.2 g. In some embodiments, MMF is administered at a dose of about 1.3 g. In some embodiments, MMF is administered at a dose of about 1.4 g. In some embodiments, MMF is administered at a dose of about 1.5 g. In some embodiments, MMF is administered at a dose of about 1.6 g. In some embodiments, MMF is administered at a dose of about 1.7 g. In some embodiments, MMF is administered at a dose of about 1.8 g. In some embodiments, MMF is administered at a dose of about 1.9 g. In some embodiments, MMF is administered at a dose of about 2 g. In some embodiments, MMF is administered at a dose of about 2.1 g. In some embodiments, MMF is administered at a dose of about 2.2 g. In some embodiments, MMF is administered at a dose of about 2.3 g. In some embodiments, MMF is administered at a dose of about 2.4 g. In some embodiments, MMF is administered at a dose of about 2.5 g.

In some embodiments, MMF is administered on each of 1, 2, 3, 4, 5, 6, 7, 8, 9, 10, or more days prior to administration of a rAAV. In some embodiments, MMF is administered on a same day as administration of a rAAV. In some embodiments, MMF is administered on each day following administration of a rAAV for about 1 week, 2 weeks, 3 weeks, 4 weeks, 1 month, 2 months, 3 months, 4 months, 5 months, 6 months, or longer. In some embodiments, MMF is administered every other day for 1, 2, 3, 4, 5, 6, 7, 8, 9, 10, or more days prior to administration of a rAAV. In some embodiments, MMF is administered on a same day as administration of a rAAV. In some embodiments, MMF is administered on every other day following administration of a rAAV for about 1 week, 2 weeks, 3 weeks, 4 weeks, 1 month, 2 months, 3 months, 4 months, 5 months, 6 months, or longer.

In some embodiments, a JAK inhibitor is administered intravenously and/or orally. In some embodiments, a JAK inhibitor is administered intravenously. In some embodiments, a JAK inhibitor is administered orally. In some embodiments, tofacitinib is administered intravenously and/or orally. In some embodiments, tofacitinib is administered intravenously. In some embodiments, tofacitinib is administered orally.

In some embodiments, a JAK inhibitor is administered every other day, once a day, twice a day, three times a day, or four times a day. In some embodiments, a JAK inhibitor is administered every other day. In some embodiments, a JAK inhibitor is administered once a day. In some embodiments, a JAK inhibitor is administered twice a day. In some embodiments, a JAK inhibitor is administered three times a day. In some embodiments, a JAK inhibitor is administered four times a day. In some embodiments, tofacitinib is administered every other day, once a day, twice a day, three times a day, or four times a day. In some embodiments, tofacitinib is administered every other day. In some embodiments, tofacitinib is administered once a day. In some embodiments, tofacitinib is administered twice a day. In some embodiments, tofacitinib is administered three times a day. In some embodiments, tofacitinib is administered four times a day.

In some embodiments, tofacitinib is administered at a dose of between about 0.01 mg/kg to about 10 mg/kg. In some embodiments, tofacitinib is administered at a dose of between about 0.1 mg/kg to about 10 mg/kg. In some embodiments, tofacitinib is administered at a dose of between about 0.1 mg/kg to about 5 mg/kg. In some embodiments, tofacitinib is administered at a dose of between about 0.1 mg/kg to about 2.5 mg/kg. In some embodiments, tofacitinib is administered at a dose of between about 0.25 mg/kg to about 2.5 mg/kg. In some embodiments, tofacitinib is administered at a dose of between about 0.1 mg/kg to about 1 mg/kg. In some embodiments, tofacitinib is administered at a dose of between about 0.5 mg/kg to about 1 mg/kg. In some embodiments, tofacitinib is administered at a dose of about 0.1 mg/kg. In some embodiments, tofacitinib is administered at a dose of about 0.15 mg/kg. In some embodiments, tofacitinib is administered at a dose of about 0.2 mg/kg. In some embodiments, tofacitinib is administered at a dose of about 0.25 mg/kg. In some embodiments, tofacitinib is administered at a dose of about 0.3 mg/kg. In some embodiments, tofacitinib is administered at a dose of about 0.35 mg/kg. In some embodiments, tofacitinib is administered at a dose of about 0.4 mg/kg. In some embodiments, tofacitinib is administered at a dose of about 0.45 mg/kg. In some embodiments, tofacitinib is administered at a dose of about 0.5 mg/kg. In some embodiments, tofacitinib is administered at a dose of about 0.55 mg/kg. In some embodiments, tofacitinib is administered at a dose of about 0.6 mg/kg. In some embodiments, tofacitinib is administered at a dose of about 0.65 mg/kg. In some embodiments, tofacitinib is administered at a dose of about 0.7 mg/kg. In some embodiments, tofacitinib is administered at a dose of about 0.75 mg/kg. In some embodiments, tofacitinib is administered at a dose of about 0.8 mg/kg. In some embodiments, tofacitinib is administered at a dose of about 0.85 mg/kg. In some embodiments, tofacitinib is administered at a dose of about 0.9 mg/kg. In some embodiments, tofacitinib is administered at a dose of about 0.95 mg/kg. In some embodiments, tofacitinib is administered at a dose of about 1 mg/kg. In some embodiments, tofacitinib is administered at a dose of about 1.1 mg/kg. In some embodiments, tofacitinib is administered at a dose of about 1.2 mg/kg. In some embodiments, tofacitinib is administered at a dose of about 1.3 mg/kg. In some embodiments, tofacitinib is administered at a dose of about 1.4 mg/kg. In some embodiments, tofacitinib is administered at a dose of about 1.5 mg/kg.

In some embodiments, tofacitinib is administered at a dose of between about 0.1 mg to about 20 mg. In some embodiments, tofacitinib is administered at a dose of between about 0.5 mg to about 15 mg. In some embodiments, tofacitinib is administered at a dose of between about 1 mg to about 10 mg. In some embodiments, tofacitinib is administered at a dose of between about 1 mg to about 5 mg. In some embodiments, tofacitinib is administered at a dose of between about 2.5 mg to about 10 mg. In some embodiments, tofacitinib is administered at a dose of between about 2.5 mg to about 5 mg. In some embodiments, tofacitinib is administered at a dose of between about 5 mg to about 10 mg. In some embodiments, tofacitinib is administered at a dose of about 1 mg. In some embodiments, tofacitinib is administered at a dose of about 2 mg. In some embodiments, tofacitinib is administered at a dose of about 3 mg. In some embodiments, tofacitinib is administered at a dose of about 4 mg. In some embodiments, tofacitinib is administered at a dose of about 5 mg. In some embodiments, tofacitinib is administered at a dose of about 6 mg. In some embodiments, tofacitinib is administered at a dose of about 7 mg. In some embodiments, tofacitinib is administered at a dose of about 8 mg. In some embodiments, tofacitinib is administered at a dose of about 9 mg. In some embodiments, tofacitinib is administered at a dose of about 10 mg.

In some embodiments, tofacitinib is administered on each of 1, 2, 3, 4, 5, 6, 7, 8, 9, 10, or more days prior to administration of a rAAV. In some embodiments, tofacitinib is administered on a same day as administration of a rAAV. In some embodiments, tofacitinib is administered on each day following administration of a rAAV for about 1 week, 2 weeks, 3 weeks, 4 weeks, 1 month, 2 months, 3 months, 4 months, 5 months, 6 months, or longer. In some embodiments, tofacitinib is administered every other day for 1, 2, 3, 4, 5, 6, 7, 8, 9, 10, or more days prior to administration of a rAAV. In some embodiments, tofacitinib is administered on a same day as administration of arAAV. In some embodiments, tofacitinib is administered on every other day following administration of a rAAV for about 1 week, 2 weeks, 3 weeks, 4 weeks, 1 month, 2 months, 3 months, 4 months, 5 months, 6 months, or longer.

### Pharmaceutical Compositions

In general, compositions comprising one or more rAAVs and compositions comprising one more immunosuppressive agents of the present disclosure may be administered in any form, including tablet, powder, or liquid, formulated into a pharmaceutically acceptable carrier or excipient, depending on a condition of a patient. Additionally, non-active ingredients well known in the art, such as binders, fillers, coatings, preservatives, coloring agents, flavoring agents, and other additives may optionally be formulated with one more administered agents described herein, or left out completely if there is a risk of negative side effects to the patient such as increased inflammation or interference with the absorption of particular compounds.

In some embodiments, the present disclosure provides pharmaceutical compositions comprising a rAAV and/or an immunosuppressive agent. In some embodiments, a pharmaceutical composition comprises one or more immunosuppressive agents. In some embodiments, a pharmaceutical composition comprises two or more immunosuppressive agents. In some embodiments, a pharmaceutical composition comprises three or more immunosuppressive agents. In some embodiments, for example, for therapeutic and clinical purposes, a rAAV and/or immunosuppressive agent is provided as a pharmaceutical composition.

In some embodiments, a pharmaceutical composition is suitable for administration or delivery of a rAAV and/or one or more immunosuppressive agents to an area or portion of a body affected by a disease, disorder, or condition. In some embodiments, a pharmaceutical composition comprises a therapeutically effective amount of a rAAV and/or immunosuppressive agent provided herein. In some embodiments, a pharmaceutical composition comprises a therapeutically effective amount of a rAAV or immunosuppressive agent, and a pharmaceutically acceptable carrier or excipient. In some embodiments, a pharmaceutically acceptable carrier is a buffer.

In some embodiments, a pharmaceutical composition is formulated for injection (e.g., intravenous injection, intrathecal injection, or subcutaneous injection), oral administration, buccal administration, inhalation, nasal administration, topical administration, ophthalmic administration, or otic administration. In some embodiments, a pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, a pharmaceutical composition is formulated for administration via a route as described herein. In some embodiments, a pharmaceutical composition is formulated for intravenous, intrathecal, intracerebroventricular, intracisternal, intramuscular, intraparenchymal, intracranial, intraocular, intra-articular, intranasal, intraosseous, intra-alveolar, intra-arterial, intraperitoneal, oral, subcutaneous, sublingual, and/or submucosal administration.

### Characterization and Assessment

In some embodiments, properties and/or activities of provided immunosuppressive agents, immunosuppressive regimens, rAAVs, and methods can be characterized and/or assessed using various technologies available to those skilled in the art, e.g., biochemical assays, cell-based assays, animal models, or clinical trials. Certain useful technologies are described in the Examples. Those skilled in the art reading the present disclosure will readily appreciate that other technologies (e.g., in vitro models (e.g., cell lines) or animal models for various diseases, disorders, or conditions) may be designed and/or utilized to assess provided technologies (e.g., immunosuppressive agents, immunosuppressive regimens, rAAVs, or methods) in accordance with the present disclosure.

### EXEMPLIFICATION

### Example 1. Immune System Responses May Play a Causal Role in Dorsal Root Ganglion Toxicity

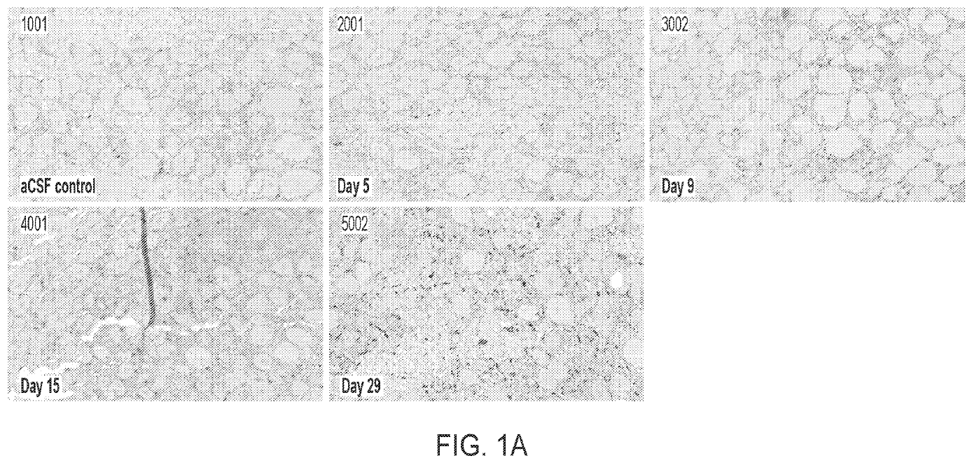

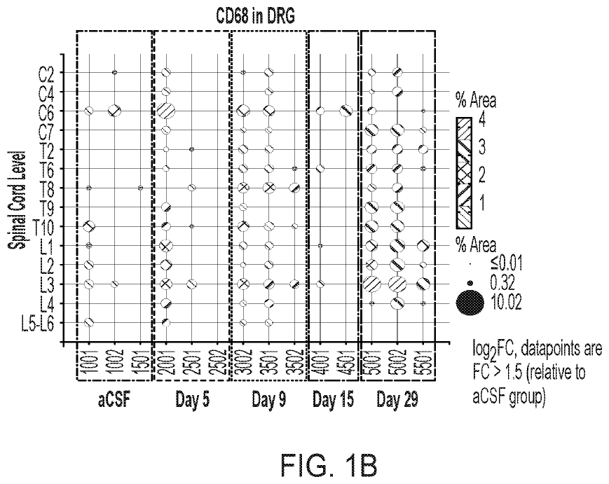

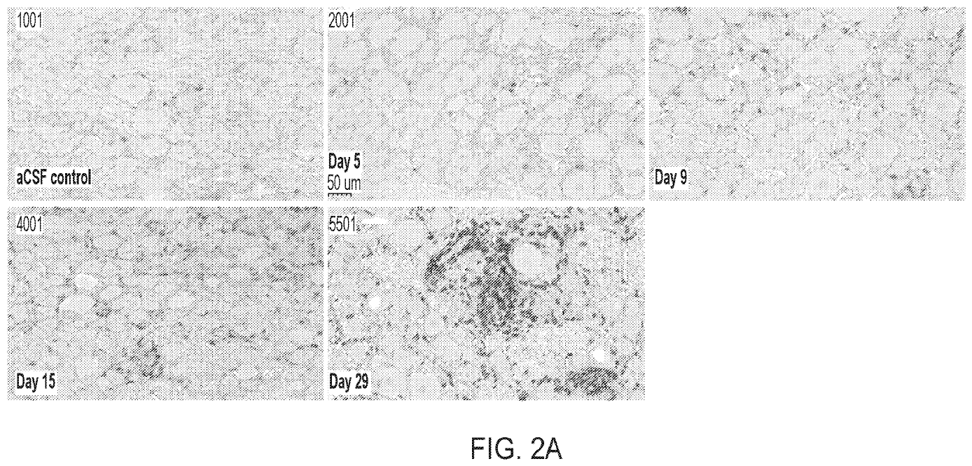

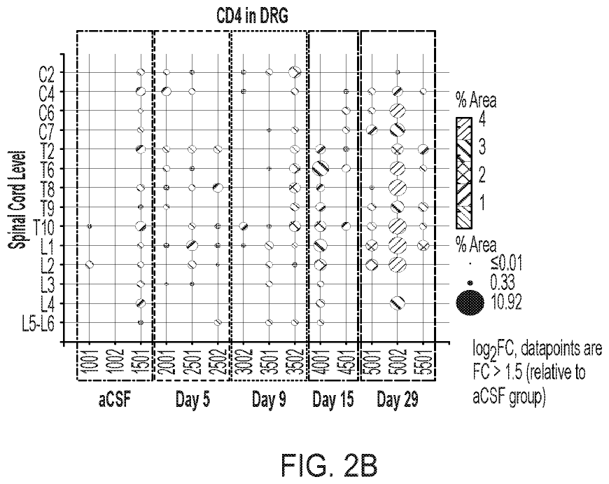

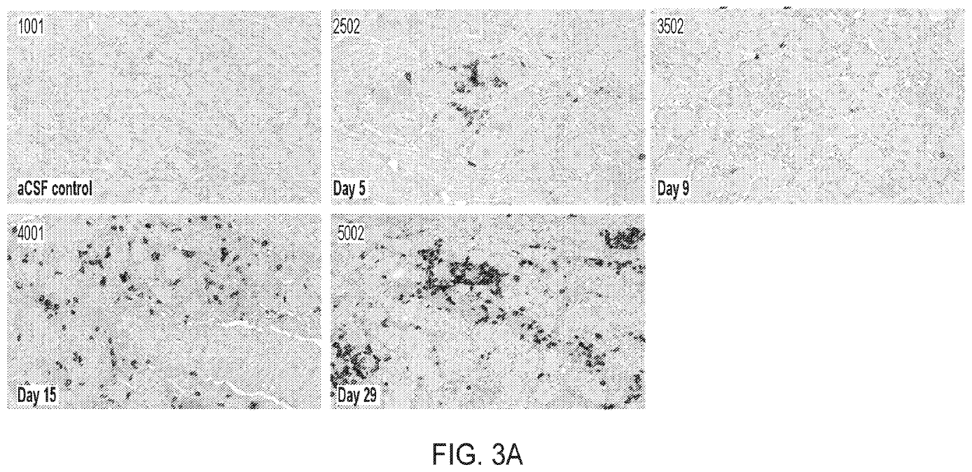

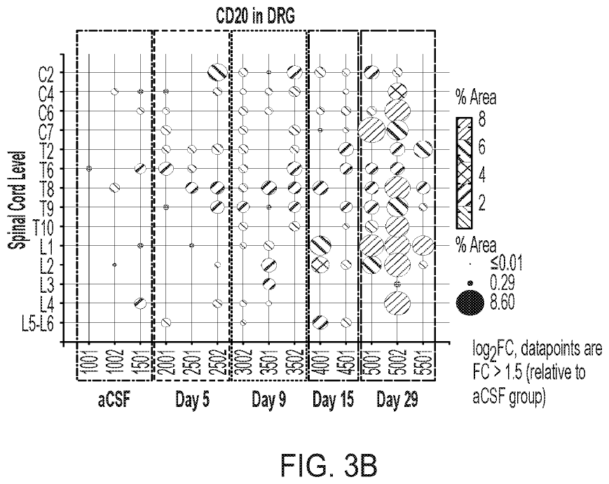

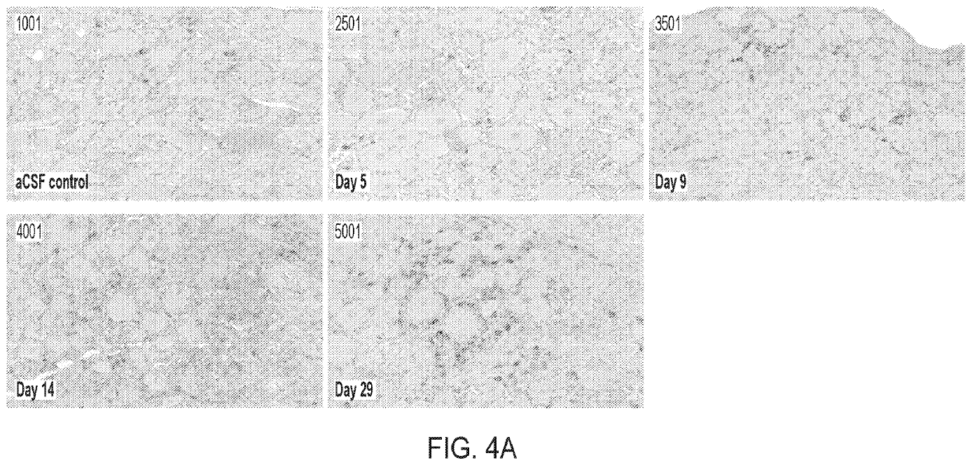

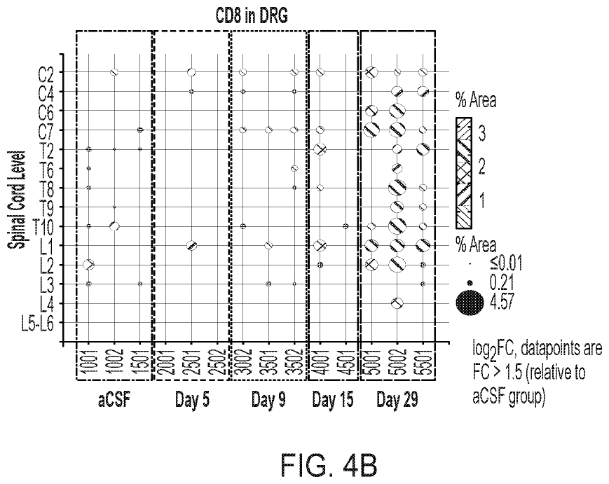

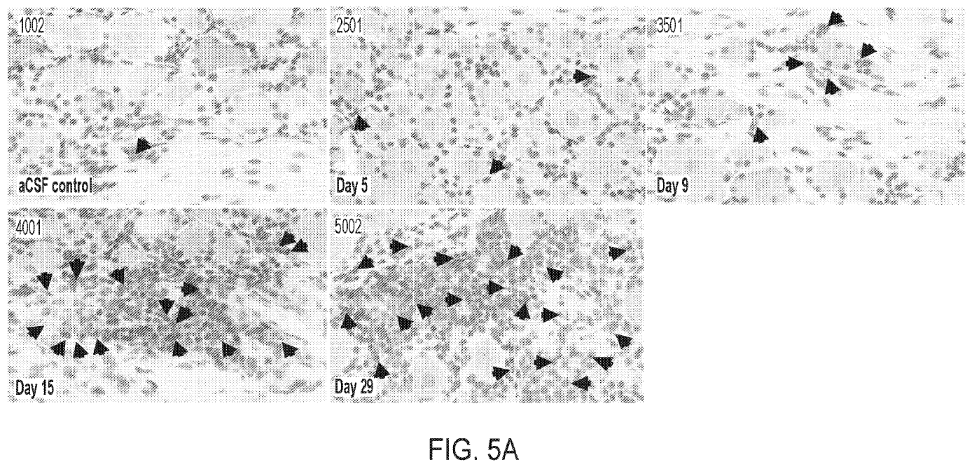

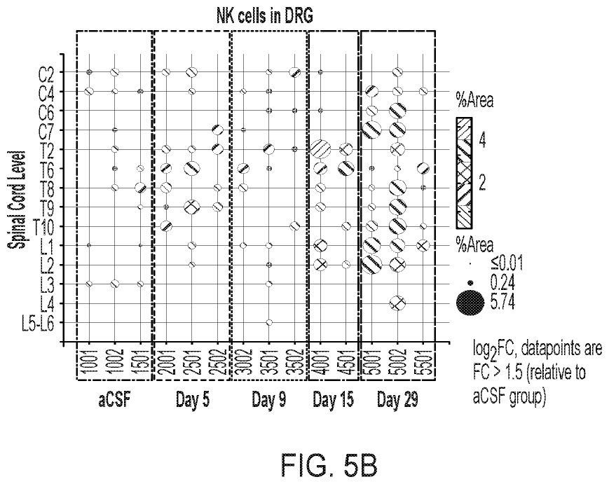

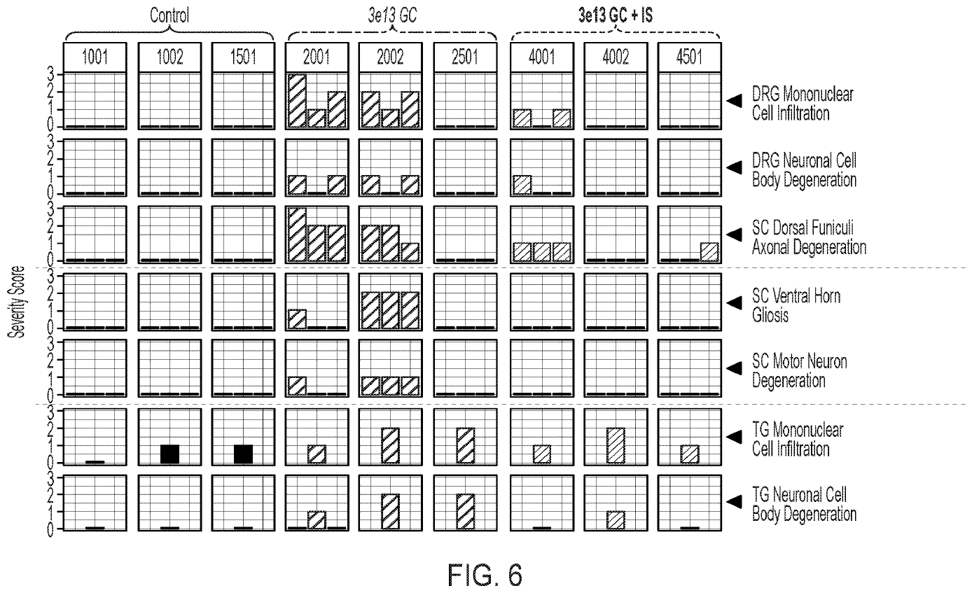

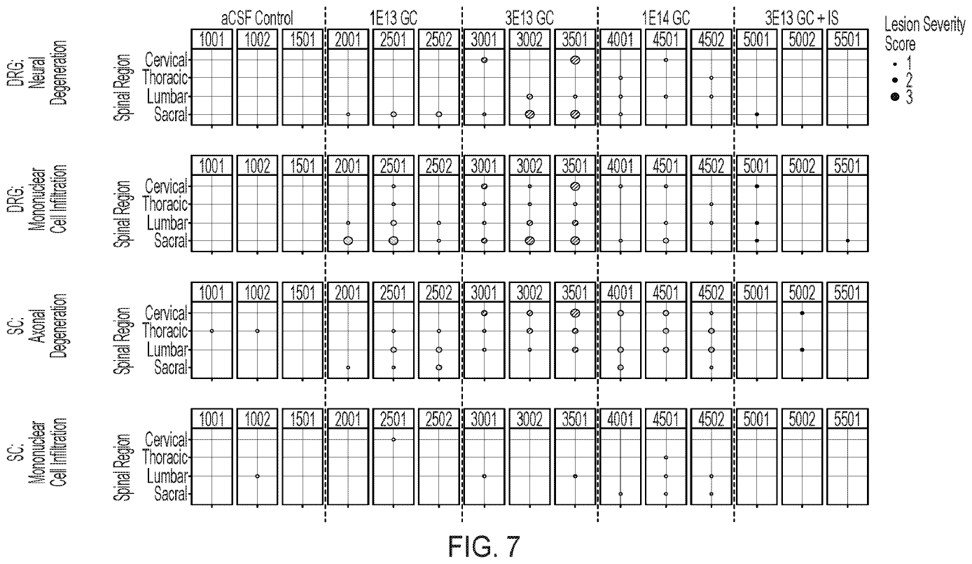

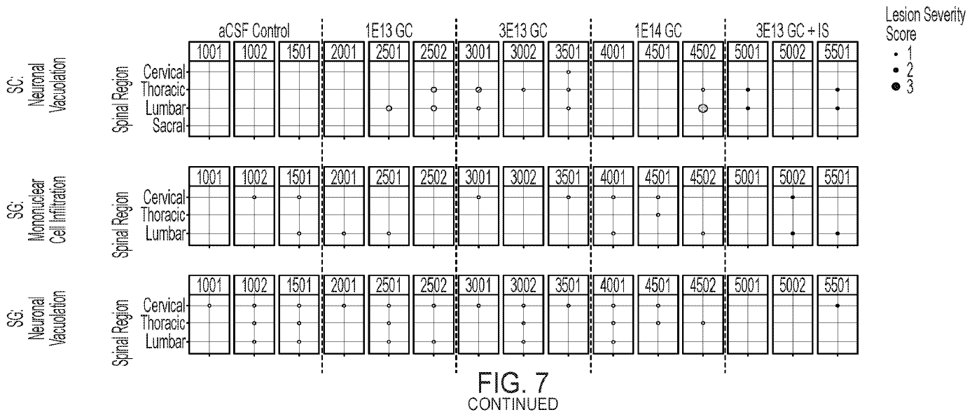

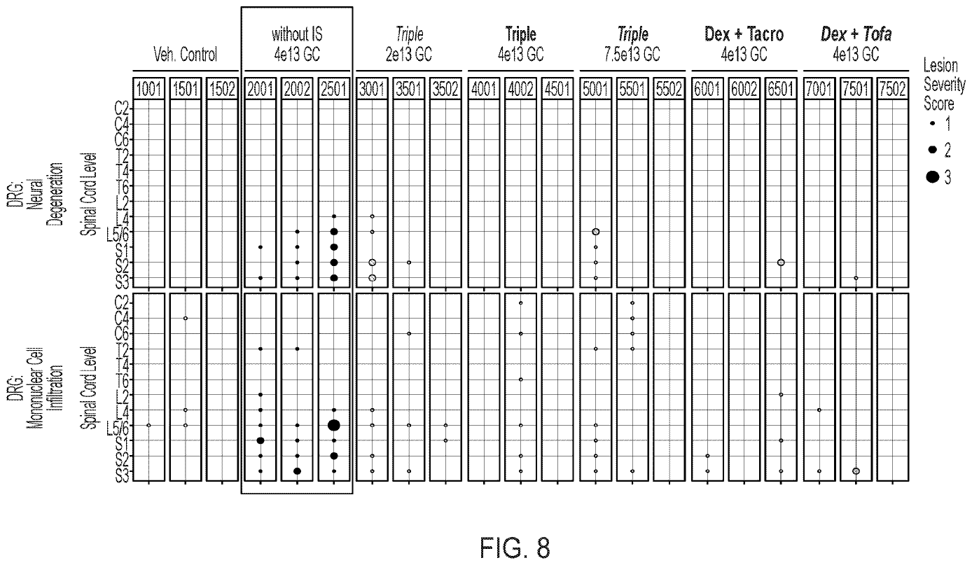

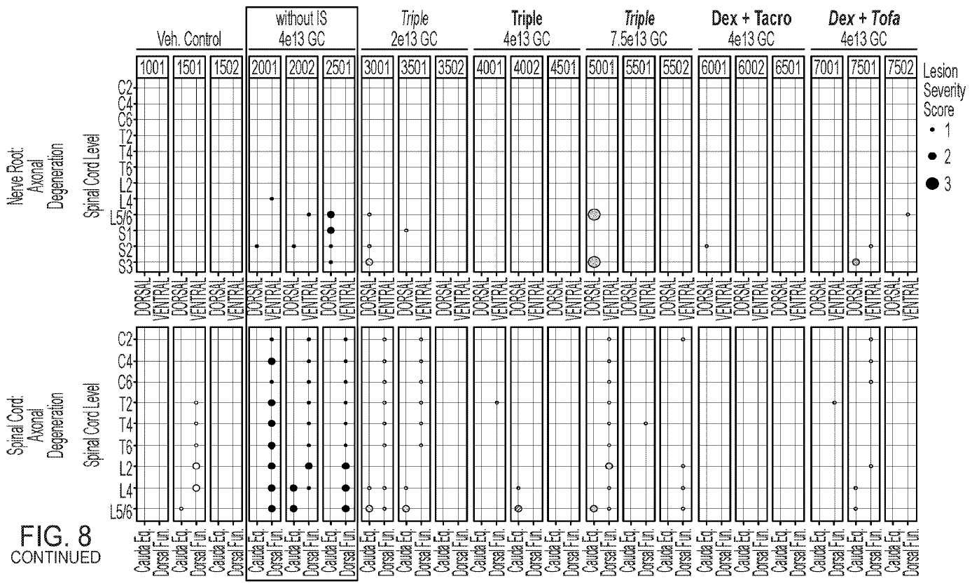

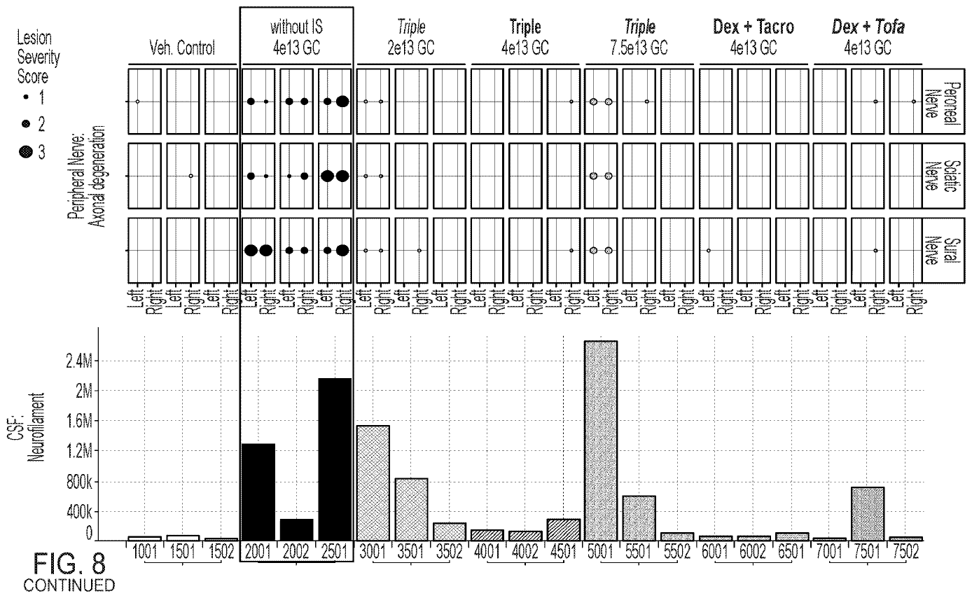

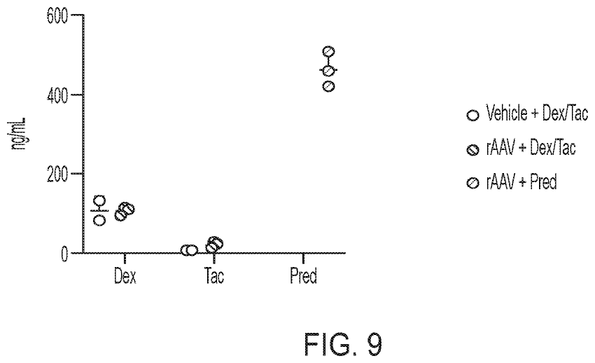

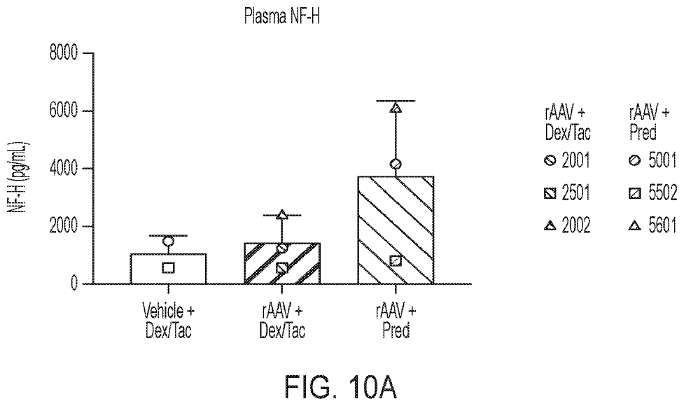

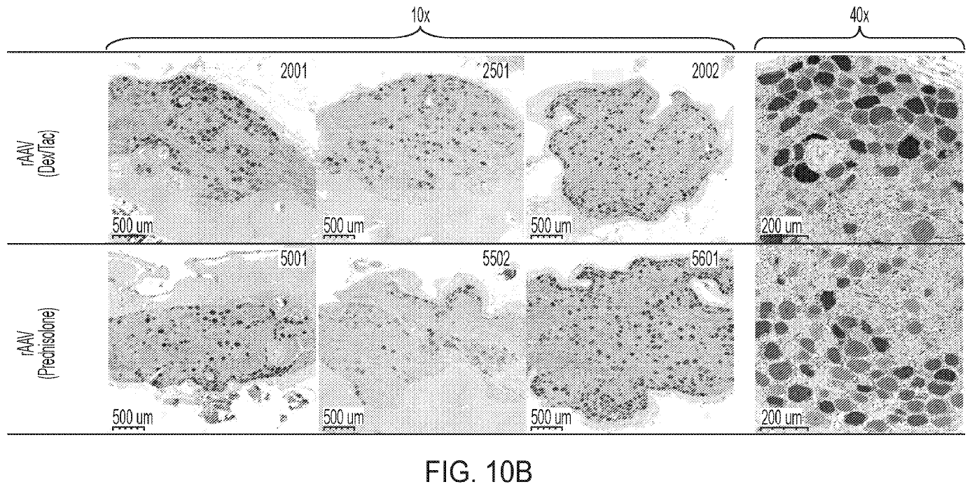

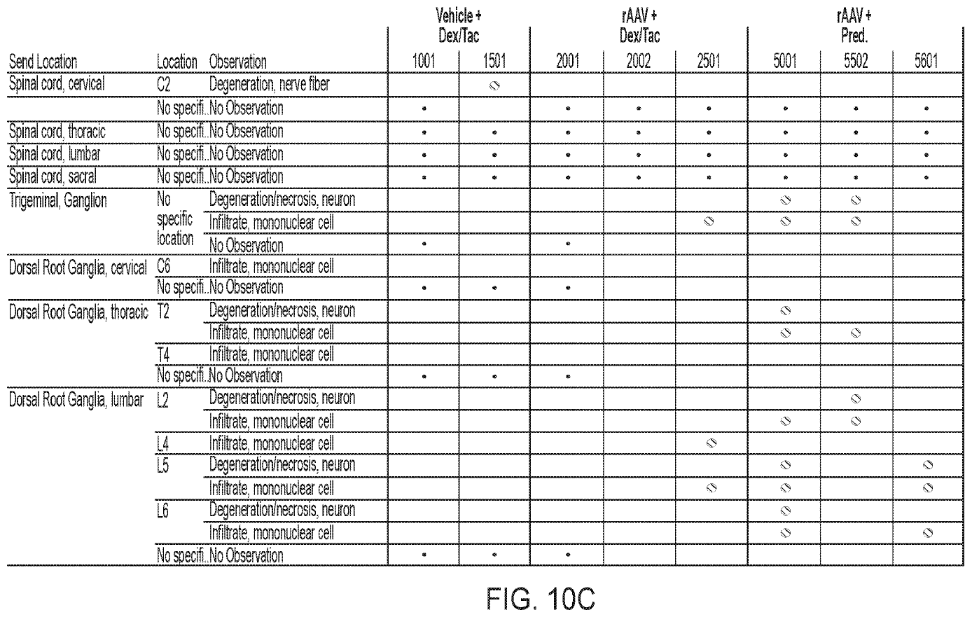

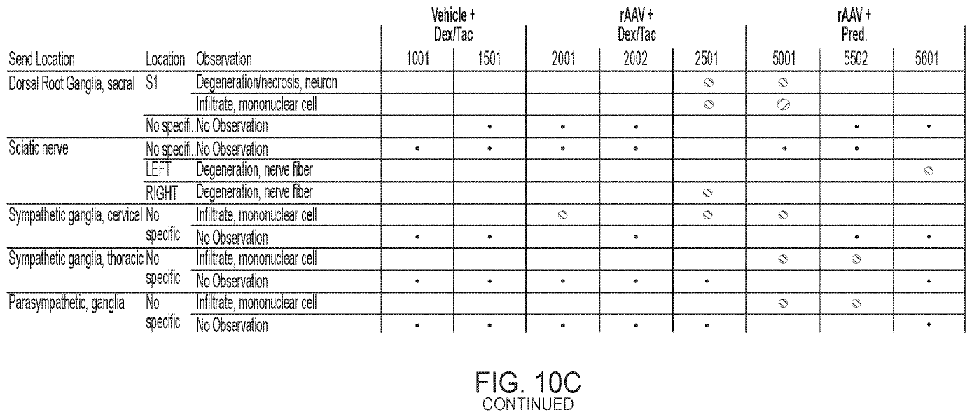

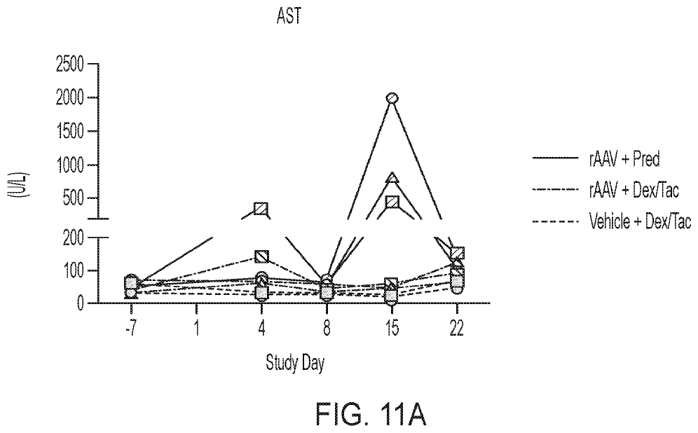

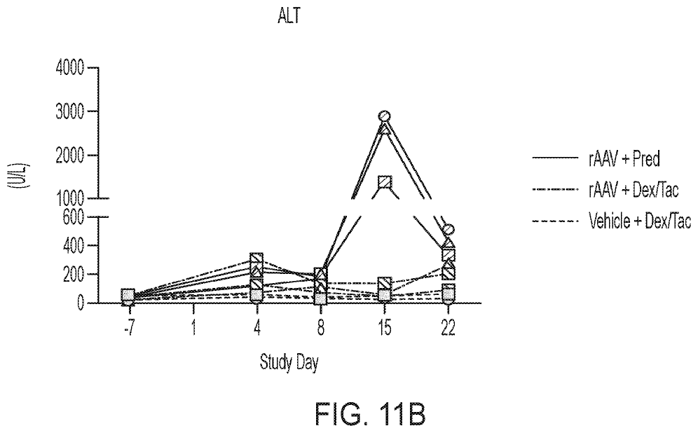

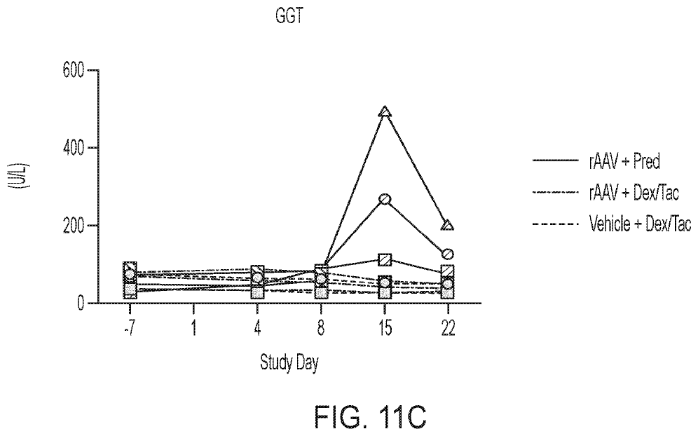

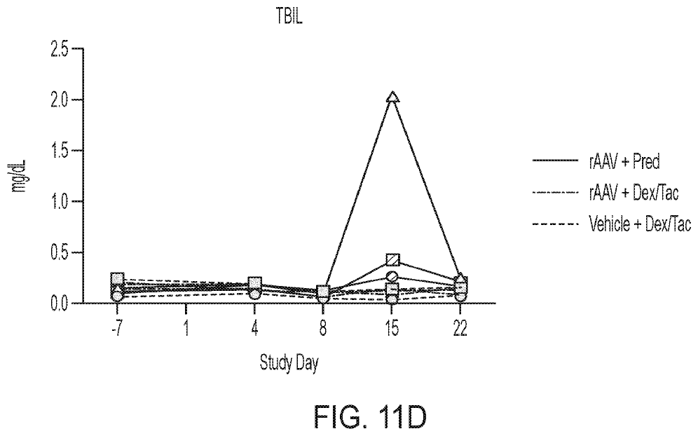

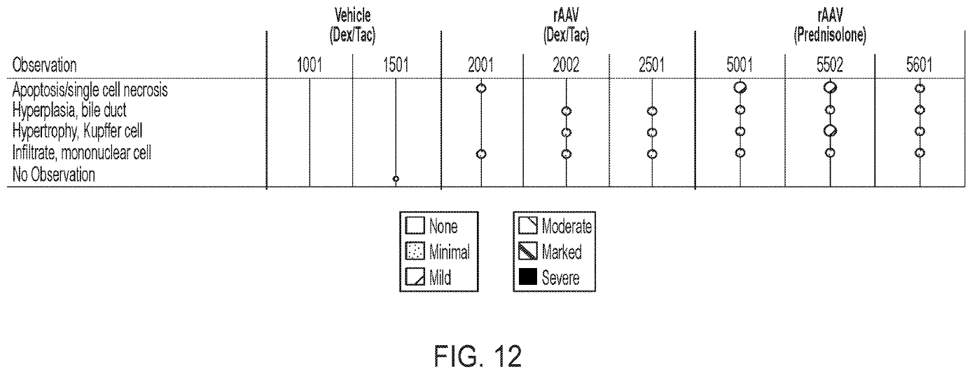

A lesion time course study was conducted to identify molecular events occurring prior to and concurrent with lesion formation. A rAAV comprising a human transgene protein-encoding nucleic acid sequence was administered to cynomolgus macaques at 3.68×10¹³ vg/animal via an intracisterna magna route. A control group of cynomolgus macaques received vehicle only. Tissues were collected at the terminal time points (days 5, 9, 15, and 29). Histopathology confirmed presence of lesions in the dorsal root ganglion (DRG), including DRG neuronal degeneration/necrosis, DRG mononuclear cell infiltration and nerve fiber degeneration in the spinal cord dorsal funiculi in animals that received the rAAV, starting at day 15 post-AAV administration and with increased severity and incidence at day 29 post-AAV administration. However, as shown in FIGS. 1-5, immunohistochemistry and in situ hybridization revealed immune cell foci were present before lesion formation, as early as day 5 (CD68+macrophages, NCR1/NKp46+natural killer cells, CD20+B cells, CD4+ T cells) or day 9 (CD8+ T cells), and increased over time (days 15 and 29) in animals that received the rAAV as compared to animals that received vehicle only (day 29). Immune cell infiltration prior to neuronal degeneration may suggest a causal role of an immune response in DRG toxicity. In situ hybridization of transgene expression identified highly transduced DRG neurons preferentially underwent neuronal degeneration and/or necrosis, suggesting that transgene expression may contribute to cell stress and/or immune cell targeting of highly transduced neurons.

### Example 2. Provided Technologies can Provide Reduction of Dorsal Root Ganglion, Trigeminal Ganglion, and Motor Neuron Toxicity

Non-human primates (2-3 year old cynomolgus macaques) were administered a rAAV, comprising a human transgene protein-encoding nucleic acid sequence, via a single intracisternal magna (ICM) injection at a dose of 3×10¹³ vg/animal on day 1. One group (n=3) of animals was administered only the rAAV. One group (n=3) of animals was administered the rAAV and an immunosuppressive regimen comprising: (i) dexamethasone (0.5 mg) via intrathecal injection on day 1. (ii) dexamethasone (0.5 mg/kg) orally once per a day starting on day-2 until study conclusion-except for day 1, (iii) tacrolimus (1 mg/kg) orally once per a day starting on day-2 until study conclusion, and (iv) mycophenolate mofetil (50 mg/kg) orally twice per a day starting on day-2 until study conclusion. A control group (n=3) of animals was not administered the rAAV or the immunosuppressive regimen.

At 4 weeks post-administration of the rAAV, animals were sacrificed and tissues were collected for histopathological analysis. DRG tissue was assessed for lesions including mononuclear cell infiltration and neuronal cell body degeneration. Spinal cord tissue was assessed for lesions including dorsal funiculi axonal degeneration, ventral horn gliosis, and motor neuron degeneration. Trigeminal ganglion tissue was assessed for lesions including mononuclear cell infiltration and neuronal cell body degeneration. Lesions were scored for severity on a scale of 0-5, where 0 represents no lesion (no tissue affected), 1 represents minimal (<10% tissue affected), 2 represents mild (10-25% tissue affected), 3 represents moderate (25-50% tissue affected), 4 represents marked (50-95% tissue affected), and 5 represents severe (>95% tissue affected). As displayed in FIG. 6, all animals in the control group were found to have minimal to no lesions in all examined tissues, whereas 2 out of 3 animals which received only the rAAV exhibited moderate to mild lesions in both DRG and spinal cord. In contrast, all animals which received the rAAV and the immunosuppressive regimen displayed minimal to no lesions across most tissues, including DRG and spinal cord. Further, mild increases in total protein and albumin in the cerebrospinal fluid (CSF) was observed for animals which received the rAAV alone as compared to the control group, while those animals that received the rAAV in combination with the immunosuppressive regimen did not display such an increase. These data suggest that this immunosuppression regimen reduces DRG and spinal cord histopathologies caused by AAV gene therapy, thus implicating a causal role for the immune response in this AAV-mediated toxicity.

### Example 3. Provided Technologies can Provide Reduction of Dorsal Root Ganglion, Spinal Cord, and Sympathetic Ganglion Toxicity

Non-human primates (cynomolgus macaques) were administered a rAAV, comprising a human transgene protein-encoding nucleic acid sequence (different than that used in Example 1 or 2), via a single intracisternal magna (ICM) injection at a dose of 3×10¹³ vg/animal on day 1. Animals were treated with an immunosuppressive regimen comprising: (i) dexamethasone (0.5 mg/kg) orally once per a day starting on day-2 until study conclusion, (ii) tacrolimus (1 mg/kg) orally once per a day starting on day-2 until study conclusion, and (iii) mycophenolate mofetil (50 mg/kg) orally twice per a day starting on day-2 until study conclusion. At study conclusion (day 43), DRG tissue was assessed for lesions including mononuclear cell infiltration and neuronal cell body degeneration. Spinal cord tissue was assessed for lesions including dorsal funiculi axonal degeneration, ventral horn gliosis, and motor neuron degeneration. Trigeminal ganglion tissue was assessed for lesions including mononuclear cell infiltration and neuronal cell body degeneration. Lesions were scored for severity on a scale of 0-5, where 0 represents no lesion (no tissue affected), 1 represents minimal (<10% tissue affected), 2 represents mild (10-25% tissue affected), 3 represents moderate (25-50% tissue affected), 4 represents marked (50-95% tissue affected), and 5 represents severe (>95% tissue affected). As displayed in FIG. 7, vehicle-treated animals had no to minimal lesions in all tissues examined, whereas 3 out of 3 animals administered rAAV (3×10¹³ vg) exhibited mild to moderate lesions in the DRG and spinal cord. In contrast, all animals treated with rAAV and the immunosuppression regimen exhibited no to minimal lesions in the DRG and spinal cord tissue. These data support the ability of an immunosuppression regimen to reduce the severity and incidence of DRG and spinal cord lesions following AAV gene therapy encoding a protein transgene.

### Example 4. Provided Technologies can Provide Reduction of Dorsal Root Ganglion, Nerve Root, Spinal Cord, and Peripheral Nerve Toxicity

Non-human primates (cynomolgus macaques) were administered a rAAV comprising a miRNA-encoding nucleic acid sequence via a single intrathecal (IT) injection via lumbar puncture (LP) at a dose of 4×10¹³ vg/animal on day 1. Animals were treated with an immunosuppressive regimen comprising: (i) dexamethasone (0.5 mg/kg) orally once per a day starting on day-2 until study conclusion and (ii) tacrolimus (1 mg/kg) orally once per a day starting on day-2 until study conclusion and/or tofacitinib (1 mg/kg) orally once per a day starting on day-2 until study conclusion. At study conclusion (day 43), DRG tissue was assessed for lesions including mononuclear cell infiltration and neuronal cell body degeneration. Spinal cord tissue was assessed for lesions including dorsal funiculi axonal degeneration, ventral horn gliosis, and motor neuron degeneration. Sciatic nerves were assessed for lesions including nerve fiber degeneration and mononuclear cell infiltration. Trigeminal ganglion tissue was assessed for lesions including mononuclear cell infiltration and neuronal cell body degeneration. Lesions were scored for severity on a scale of 0-5, where 0 represents no lesion (no tissue affected), 1 represents minimal (<10% tissue affected), 2 represents mild (10-25% tissue affected), 3 represents moderate (25-50% tissue affected), 4 represents marked (50-95% tissue affected), and 5 represents severe (>95% tissue affected). As displayed in FIG. 8, all vehicle-treated animals exhibited no to minimal lesions in all tissues examined, whereas 3 out of 3 animals administered 4×10¹³ vg/animal rAAV exhibited mild to moderate lesions in both the DRG and spinal cord. In contrast, the incidence of lesions in animals administered the 4×10¹³ vg rAAV in combination with the “triple” (dexamethasone, tacrolimus, and tofacitinib) immunosuppression regimen was reduced in both the spinal cord and DRGs in 3 out of 3 animals; the severity was also reduced. Lesion incidence and severity was also decreased in animals administered 4×10¹³ vg/animal rAAV in combination with an immunosuppression regimen of dexamethasone and tacrolimus or dexamethasone and tofacitinib. Collectively, these data support a causal role of the immune response in DRG and spinal cord lesion formation in response to AAV gene therapy encoding a miRNA transgene and demonstrate that lesion incidence and severity can be reduced when rAAVs are administered in combination with an immunosuppression regimen.

### Example 5. Provided Technologies can Provide Reduction of Toxicity In Vivo

Non-human primates (cynomolgus macaques) were administered 5×10¹³ vg/kg of rAAV, comprising a nucleic acid sequence encoding mCherry protein, via a single intravenous (IV) injection, on day 0, in combination with an immunosuppression regimen comprising dexamethasone and tacrolimus (Dex/Tac) (N=3) or an immunosuppression regimen comprising prednisolone (Pred) (N=3). A control group of animals (N=2) received a vehicle control in combination with an immunosuppression regimen comprising dexamethasone and tacrolimus. Dexamethasone was administered orally once per day at a dosage of 0.5 mg/kg starting on day-2 through day 21; tacrolimus was administered orally once per day at a dosage of 1 mg/kg starting on day-2 through day 21; prednisolone was administered orally once per day at a dosage of 3 mg/kg starting on day-2 through day 21. Blood and plasma samples were collected from animals at various time points to assess peripheral immunosuppression drug exposures, levels of plasma neurofilament H, and levels of liver enzymes (AST, ALT, ALP, GGT, LDH) and total bilirubin (TBIL). As shown in FIG. 9, the implemented immunosuppression regimens (Dex/Tac or Pred) achieved targeted ranges of peripheral drug exposure for each drug (Dex, Tac, or Pred) as examined on day 21. Animals were sacrificed at end of study (day 22) and tissues collected and examined for lesions. Certain exemplary data are shown in FIG. 10, FIG. 11, and FIG. 12, and Table 1 and Table 2 below.

An immunosuppression regimen comprising dexamethasone and tacrolimus was capable of reducing neuronal toxicity of treatment with a rAAV. As displayed in FIG. 10A, levels of plasma NF—H was decreased in animals administered an immunosuppression regimen comprising Dex/Tac in combination with rAAV as compared to animals administered an immunosuppression regimen comprising Pred in combination with rAAV. Furthermore, animals administered an immunosuppression regimen comprising Dex/Tac in combination with rAAV exhibited comparable levels of plasma NF—H as compared to animals administered an immunosuppression regimen comprising Dex/Tac in combination with vehicle only. These results indicate that administration of an immunosuppression regimen comprising Dex/Tac can provide reduction of neuronal toxicity from treatment with a rAAV, and can provide a greater reduction as compared to administration of an immunosuppression regimen comprising Pred. Additionally, examination of tissues from treated animals confirmed that level of expression of mCherry from the rAAV related to the level of plasma NF—H observed in animals administered Pred. In contrast, the level of expression of mCherry from the rAAV did not relate to the level of plasma NF—H observed in animals administered Dex/Tac. Exemplary immunohistochemistry images displaying mCherry expression can be seen in FIG. 10B. In some embodiments, an immunosuppression regimen provided herein, e.g., an immunosuppression regimen comprising dexamethasone and tacrolimus, can provide reduced neuronal toxicity in a subject upon treatment with a rAAV as compared to a different immunosuppression regimen, e.g., an immunosuppression regimen not comprising dexamethasone and tacrolimus and/or comprising prednisolone.

As shown in FIG. 10C and Table 1 below, animals administered an immunosuppression regimen comprising Dex/Tac in combination with rAAV exhibited reduced incidence and severity of histopathological lesions in several tissues, including neuronal tissues, e.g., dorsal root ganglia (DRG), and heart tissue. In various examined tissues, animals administered an immunosuppression regimen comprising Dex/Tac in combination with rAAV showed comparable incidence and/or severity of histopathological lesions as compared to animals administered the same immunosuppression but in combination with vehicle only. In some embodiments, an immunosuppression regimen provided herein, e.g., an immunosuppression regimen comprising dexamethasone and tacrolimus, can provide reduced incidence and/or severity of lesions in DRG in a subject upon treatment with a rAAV as compared to a different immunosuppression regimen, e.g., an immunosuppression regimen not comprising dexamethasone and tacrolimus and/or comprising prednisolone. In some embodiments, an immunosuppression regimen provided herein, e.g., an immunosuppression regimen comprising dexamethasone and tacrolimus, can provide reduced incidence and/or severity of lesions in heart tissue in a subject upon treatment with a rAAV as compared to a different immunosuppression regimen, e.g., an immunosuppression regimen not comprising dexamethasone and tacrolimus and/or comprising prednisolone.

TABLE 1
Histopathological lesions in examined animals.

| | Treatment | | |
|---|---|---|---|
| | Vehicle | rAAV | rAAV |
| | Immunosuppression Regimen | | |
| | Dex/Tac | Dex/Tac | Pred |
| No. Animals Examined | 2 | 3 | 3 |
| Trigeminal ganglion (No. examined) | 2 | 3 | 3 |
| Degeneration/necrosis, neuron | (0) | (0) | (2) |
| Minimal | 0 | 0 | 2 |
| Infiltrate, mononuclear cell | (0) | (1) | (2) |
| Minimal | 0 | 1 | 2 |
| Dorsal root ganglia, cervical (No. examined) | 2 | 3 | 3 |
| Infiltrate, mononuclear cell | (0) | (0) | (0) |
| Minimal | 0 | 0 | 0 |
| Dorsal root ganglia, thoracic (No. examined) | 2 | 3 | 3 |
| Degeneration/necrosis, neuron | (0) | (0) | (1) |
| Minimal | 0 | 0 | 1 |
| Infiltrate, mononuclear cell | (0) | (0) | (2) |
| Minimal | 0 | 0 | 2 |
| Dorsal root ganglia, lumbar (No. examined) | 2 | 3 | 3 |
| Degeneration/necrosis, neuron | (0) | (0) | (3) |
| Minimal | 0 | 0 | 3 |
| Infiltrate, mononuclear cell | (0) | (1) | (3) |
| Minimal | 0 | 1 | 3 |
| Dorsal root ganglia, sacral (No. examined) | 2 | 3 | 3 |
| Degeneration/necrosis, neuron | (0) | (1) | (1) |
| Minimal | 0 | 1 | 1 |
| Infiltrate, mononuclear cell | (0) | (1) | (1) |
| Minimal | 0 | 1 | 0 |
| Mild | 0 | 0 | 1 |
| Sciatic nerve (No. examined) | 2 | 3 | 3 |
| Degeneration, nerve fiber | (0) | (1) | (1) |
| Minimal | 0 | 1 | 1 |
| Sympathetic ganglia, cervical (No. examined) | 1 | 3 | 2 |
| Infiltrate, mononuclear cell | (0) | (2) | (1) |
| Minimal | 0 | 2 | 1 |
| Sympathetic ganglia, thoracic (No. examined) | 2 | 1 | 3 |
| Infiltrate, mononuclear cell | (0) | (0) | (2) |
| Minimal | 0 | 0 | 2 |
| Heart (No. examined) | 2 | 3 | 3 |
| Infiltrate, mononuclear cell, myocardium | (0) | (3) | (2) |
| Minimal | 0 | 3 | 1 |
| Mild | 0 | 0 | 1 |
| Degeneration, myocardium | (0) | (0) | (1) |
| Minimal | 0 | 0 | 1 |
| Infiltrate, mononuclear cell, adipose | (0) | (0) | (3) |
| Minimal | 0 | 0 | 3 |

Reduction in liver toxicity upon treatment with rAAV by tested immunosuppression regimens was investigated through measurement of levels of liver enzymes and analysis of liver lesions at necropsy. As depicted in FIG. 11 and Table 2 below, AAV-mediated increases in liver enzymes were reduced in animals administered an immunosuppression regimen comprising Dex/Tac in combination with rAAV as compared to animals administered an immunosuppression regimen comprising Pred in combination with rAAV. Increases in levels of aspartate aminotransferase (AST), alanine aminotransferase (ALT), and gamma-glutamyl transferase (GGT) were particularly reduced with usage of Dex/Tac as compared to usage of Pred (see FIG. 11A, FIG. 11B, FIG. 11C, and Table 2). The greatest fold-change from baseline for individual animals for all liver enzymes was greater in animals administered Pred versus animals administered Dex/Tac (Table 2). Total bilirubin levels were also examined in all animals as shown in FIG. 11D and Table 2, with levels within normal limits in animals administered Dex/Tac and notable increases in animals administered Pred. In some embodiments, an immunosuppression regimen provided herein, e.g., an immunosuppression regimen comprising dexamethasone and tacrolimus, can provide reduced increases in levels of liver enzymes, e.g., from baseline, in a subject upon treatment with a rAAV as compared to a different immunosuppression regimen, e.g., an immunosuppression regimen not comprising dexamethasone and tacrolimus and/or comprising prednisolone. In some embodiments, an immunosuppression regimen provided herein, e.g., an immunosuppression regimen comprising dexamethasone and tacrolimus, can provide a reduced increase in level of total bilirubin, e.g., from baseline, in a subject upon treatment with a rAAV as compared to a different immunosuppression regimen, e.g., an immunosuppression regimen not comprising dexamethasone and tacrolimus and/or comprising prednisolone.

As with the increases in levels of liver enzymes and total bilirubin, liver lesions were reduced in animals administered an immunosuppression regimen comprising Dex/Tac in combination with rAAV as compared to animals administered an immunosuppression regimen comprising Pred in combination with rAAV. Observed liver lesions and severity thereof in individual animals is shown in FIG. 12. Both incidence and severity of liver lesions were reduced in animals administered an immunosuppression regimen comprising Dex/Tac in combination with rAAV as compared to animals administered an immunosuppression regimen comprising Pred in combination with rAAV. In some embodiments, an immunosuppression regimen provided herein, e.g., an immunosuppression regimen comprising dexamethasone and tacrolimus, can provide reduced incidence and/or severity of lesions in liver tissue in a subject upon treatment with a rAAV as compared to a different immunosuppression regimen, e.g., an immunosuppression regimen not comprising dexamethasone and tacrolimus and/or comprising prednisolone.

TABLE 2
Range of greatest fold-change from baseline for
liver enzymes for animals within each group.

| Treatment | Vehicle | rAAV | rAAV |
|---|---|---|---|
| Immunosuppression Regimen | Dex/Tac | Dex/Tac | Pred |
| AST (U/L) | 1.15x-1.53x | 3.71x-3.93x | 10.31x-39.86x |
| ALT (U/L) | 1.28x-1.83x | 2.93x-10.96x | 25.83x-76.31x |
| ALP (U/L) | 0.75x-1.39x | 0.93x-0.68x | 2.06x-3.74x |
| GGT (U/L) | 0.67x-0.81x | WNL | 3.60x-9.48x |
| LDH (U/L) | 0.55x-1.47x | 1.14x-2.01x | 1.69x-2.31x |
| TBIL (mg/dL) | 0.48x-1.50x | WNL | 2.89x-13.73x |

AST = aspartate aminotransferase; ALT = alanine aminotransferase; ALP = alkaline phosphatase; GGT = gamma-glutamyl transferase; LDH = lactate dehydrogenase; TBIL = total bilirubin; WNL = within normal limits.

### EQUIVALENTS

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. A method of treating a subject, the method comprising:
(a) administering a recombinant adeno-associated viral vector (rAAV); and
(b) administering an immunosuppressive regimen,
wherein the immunosuppressive regimen comprises:
(i) dexamethasone and
(ii) a calcineurin inhibitor.

2. The method of claim 1, wherein the calcineurin inhibitor comprises a macrolide.

3. The method of claim 1 or 2, wherein the calcineurin inhibitor comprises tacrolimus.

4. The method of any one of the preceding claims, wherein the rAAV comprises a nucleic acid sequence encoding a polypeptide.

5. The method of any one of the preceding claims, wherein the rAAV comprises a nucleic acid sequence encoding an RNA molecule.

6. The method of any one of the preceding claims, wherein the rAAV is administered intravenously, intrathecally, intracerebroventricularly, intracisternally, intramuscularly, intraparenchymally, intracranially, intraocularly, intraarticularly, intranasally, and/or subcutaneously.

7. The method of claim 6, wherein the rAAV is administered intravenously.

8. The method of any one of the preceding claims, wherein dexamethasone is administered orally, intrathecally, and/or intravenously.

9. The method of claim 8, wherein the dexamethasone is administered orally.

10. The method of any one of the preceding claims, wherein the dexamethasone is administered at a dose of between about 0.01 mg/kg to about 10 mg/kg, about 0.1 mg/kg to about 5 mg/kg, about 0.25 mg/kg to about 2.5 mg/kg, or about 0.5 mg/kg to about 1 mg/kg.

11. The method of any one of the preceding claims, wherein the dexamethasone is administered daily.

12. The method of any one of the preceding claims, wherein the dexamethasone is administered:
(i) on each of 1, 2, 3, 4, 5, 6, 7, 8, 9, 10, or more days prior to administration of the rAAV;
(ii) on a same day as administration of the rAAV; and/or
(iii) on each day following administration of the rAAV for about 1 week, 2 weeks, 3 weeks, 4 weeks, 1 month, 2 month, 3 month, 4 month, 5 month, 6 months, or longer.

13. The method of any one of the preceding claims, wherein the calcineurin inhibitor is administered orally and/or intravenously.

14. The method of claim 13, wherein the calcineurin inhibitor is administered orally.

15. The method of any one of the preceding claims, wherein the calcineurin inhibitor is administered at a dose of between about 0.01 mg/kg to about 10 mg/kg, about 0.1 mg/kg to about 5 mg/kg, about 0.25 mg/kg to about 2.5 mg/kg, or about 0.5 mg/kg to about 1 mg/kg.

16. The method of any one of the preceding claims, wherein the calcineurin inhibitor is administered daily.

17. The method of any one of the preceding claims, wherein the calcineurin inhibitor is administered:
(i) on each of 1, 2, 3, 4, 5, 6, 7, 8, 9, 10, or more days prior to administration of the rAAV;
(ii) on a same day as administration of the rAAV; and/or
(iii) on each day following administration of the rAAV for about 1 week, 2 weeks, 3 weeks, 4 weeks, 1 month, 2 month, 3 month, 4 month, 5 month, 6 months, or longer.

18. The method of any one of the preceding claims, wherein the subject is a human.

19. The method of claim 18, wherein the subject is an adult.

20. The method of claim 18, wherein the subject is a child.

21. The method of any one of the preceding claims, wherein dorsal root ganglion (DRG) toxicity is reduced relative to administration of the rAAV without the immunosuppressive regimen.

22. The method of any one of the preceding claims, wherein lesion severity and/or incidence in one or more DRG is reduced relative to administration of the rAAV without the immunosuppressive regimen.

23. The method of any one of the preceding claims, wherein nerve fiber degeneration in one or more DRG is reduced relative to administration of the rAAV without the immunosuppressive regimen.

24. The method of any one of the preceding claims, wherein axonal degeneration in one or more DRG is reduced relative to administration of the rAAV without the immunosuppressive regimen.

25. The method of any one of the preceding claims, wherein neuronal cell body degeneration and/or necrosis in one or more DRG is reduced relative to administration of the rAAV without the immunosuppressive regimen.

26. The method of any one of the preceding claims, wherein mononuclear cell infiltration in one or more DRG is reduced relative to administration of the rAAV without the immunosuppressive regimen.

27. The method of any one of the preceding claims, wherein the immunosuppressive regimen further comprises an inosine monophosphate dehydrogenase (IMPDH) inhibitor and/or a Janus kinase (JAK) inhibitor.

28. The method of claim 27, wherein the IMPDH inhibitor comprises mycophenolate mofetil (MMF).

29. The method of claim 28, wherein MMF is administered orally or intravenously.

30. The method of claim 28 or 29, wherein MMF is administered at a dose of between about 0.1 mg/kg to about 200 mg/kg, about 1 mg/kg to about 100 mg/kg, about 10 mg/kg to about 75 mg/kg, or about 25 mg/kg to about 50 mg/kg.

31. The method of any one of claims 28-30, wherein MMF is administered daily or twice daily.

32. The method of any one of claims 28-31, wherein MMF is administered:
(i) on each of 1, 2, 3, 4, 5, 6, 7, 8, 9, 10, or more days prior to administration of the rAAV;
(ii) on a same day as administration of the rAAV; and/or
(iii) on each day following administration of the rAAV for about 1 week, 2 weeks, 3 weeks, 4 weeks, 1 month, 2 month, 3 month, 4 month, 5 month, 6 months, or longer.

33. The method of claim 27, wherein the JAK inhibitor comprises tofacitinib.

34. The method of claim 33, wherein tofacitinib is administered orally or intravenously.

35. The method of claim 33 or 34, wherein tofacitinib is administered at a dose of between about 0.01 mg/kg to about 10 mg/kg, about 0.1 mg/kg to about 5 mg/kg, about 0.25 mg/kg to about 2.5 mg/kg, or about 0.5 mg/kg to about 1 mg/kg.

36. The method of any one of claims 33-35, wherein tofacitinib is administered daily.

37. The method of any one of claims 33-36, wherein tofacitinib is administered:
(i) on each of 1, 2, 3, 4, 5, 6, 7, 8, 9, 10, or more days prior to administration of the rAAV;
(ii) on a same day as administration of the rAAV; and/or
(iii) on each day following administration of the rAAV for about 1 week, 2 weeks, 3 weeks, 4 weeks, 1 month, 2 month, 3 month, 4 month, 5 month, 6 months, or longer.
